SEQUENCE LISTING (6 sequences)

SEQ ID NO: 1 - protein, 404 aa, synthetic construct
MTTTEETPRLPFARANALAIAPDYEVLRSRAPISRVLTPAGDPAWLVTSYEEAKEVFRDKRFGRSHPAPEQASVISNAAIQDGPSGDFETEEAEHKRMRRMLAPAFSAPRMRALGDRIAELTDRCLDDLQAAHDANPGAPVDLTDFLAFPLPVLVICELLGVPYADREHFRGLSERIARMDGGEDAQAAMAEFKAYMLKLAEAKRADPQPDVISDLVAVQADDPTFTDDDLSRMATGLLFAGHETTSTRIAMGTLFLLSDTARRDRFAADPDGEVNQTVEEILRLTATSGTGLLRYAHEDVEIAGTRIRRGDAVLVSSDAANRDASVFADPDEFDPGRTPNVHLAFGTGAHVCIGANLARTELRTVFPKLFRRFPGLRLAAGIEDVPVRVNRVAGGVDAVPVTW

SEQ ID NO: 2 - DNA, 1212 bp, synthetic construct
  source 1..1212: mol_type = other DNA
atgaccaccaccgaagaaaccccgcgtctgccgtttgcacgtgcgaacgcgctggcaatcgctccggattacgaagtgctgcgcagccgtgcgccgatctcccgtgttctgaccccggcgggcgacccggcttggctggttaccagctacgaagaagcgaaagaagtgttccgtgataaacgctttggccgtagccatccggcgccggaacaggcgagcgttatcagcaacgcggcgatccaggacggcccgagcggcgacttcgaaaccgaagaagcggaacacaaacgtatgcgtcgtatgttagcaccggcattcagcgcgccgcgtatgcgtgctctgggtgatcgcatcgcagaattgaccgatcgttgcctggatgatctgcaggcggcacacgacgcgaatccgggtgcgccggttgatctgactgatttcctggcgttcccgctgccggttctggtgatctgcgaactgctgggtgttccgtacgcggaccgcgaacacttccgtggcctgtctgaacgcatcgcgcgcatggacggtggtgaggacgcgcaggcagctatggcggaattcaaagcgtacatgctgaaactggctgaagcgaaacgtgctgatccgcagccggacgtgattagcgatctggtcgcagttcaggcggatgacccgaccttcaccgatgacgacctgagccgtatggcaaccggtctgctgttcgcgggtcacgaaaccaccagcacccgcatcgctatgggcactctgtttctgctgtctgataccgctcgtcgtgatcgcttcgcagcggacccggatggcgaagttaaccagaccgttgaagaaattctgcgtctgaccgcgacctctggcaccggtctgctgcgttacgcgcatgaggatgttgaaatcgcaggcacccgcattcgtcgtggtgatgcggtgctggttagctctgatgcggcgaaccgtgatgcgtctgttttcgcggacccggatgaatttgatccgggtcgtaccccgaacgtgcacctggcgttcggtaccggcgcgcacgtttgtatcggcgcgaacctggcgcgtaccgaactgcgtaccgtgttcccgaaactgttccgtcgcttcccaggtctgcgtctggcggctggcatcgaagatgttccggttcgtgttaaccgtgtggcgggcggtgttgatgcggttccggttacctgg

SEQ ID NO: 3 - protein, 329 aa, synthetic construct
VLHRHQPVTIGEPAARAVSRTVTVERLDRIADDVLRLVLRDAGGKTLPTWTPGAHIDLDLGALSRQYSLCGAPDAPSYEIAVHLDPESRGGSRYIHEQLEVGSPLRMRGPRNHFALDPGAEHYVFVAGGIGITPVLAMADHARARGWSYELHYCGRNRSGMAYLERVAGHGDRAALHVSEEGTRIDLAALLAEPAPGVQIYACGPGRLLAGLEDASRNWPDGALHVEHFTSSLAALDPDVEHAFDLELRDSGLTVRVEPTQTVLDALRANNIDVPSDCEEGLCGSCEVAVLDGEVDHRDTVLTKAERAANRQMMTCCSRACGDRLALRL

SEQ ID NO: 4 - DNA, 987 bp, synthetic construct
  source 1..987: mol_type = other DNA
gtgctgcatcgtcaccagccggtgaccattggcgaacccgcagcccgtgcagtttcgcgcactgttaccgtggaacgtctggatcgtatcgctgacgatgtacttcgtcttgtactgcgtgatgcgggcggcaaaaccttacctacatggactcctggagcgcacatcgatctggatcttggagctttgtctcgccaatacagcctttgtggggctccggacgccccttcgtatgaaattgcagtccatcttgacccagagtcacgtggcggaagtcgctatatccatgagcagttggaagttggctctccgcttcgtatgcgtggacctcgcaaccattttgcgcttgatccaggggctgagcattatgtctttgttgccgggggaatcgggatcacgccagtattagcgatggctgaccatgctcgtgcccgtggttggtcgtatgagcttcactattgtggacgtaatcgtagcggaatggcctatcttgagcgcgtcgcaggccacggggatcgtgctgcacttcacgtaagcgaggagggcactcgtattgacttagcagctctgttggcggaacctgcccccggagtacaaatttacgcttgcggaccgggacgcttactggctgggctggaggatgcatcacgtaactggccagacggcgcactgcatgtagagcatttcacatcgagcctggctgctctggaccccgacgttgagcatgcctttgatttggaacttcgtgactccggcctgaccgtgcgtgtggaacctacccaaacagtattagacgctcttcgcgccaataatatcgacgttccctccgactgtgaggaggggctttgtggatcttgcgaggtggcagtgcttgacggagaggttgatcatcgtgacaccgtccttacgaaagccgaacgtgcagcgaaccgccaaatgatgacgtgttgctcgcgtgcctgtggagaccgcttggcattacgtctt

SEQ ID NO: 5 - protein, 335 aa, synthetic construct
LPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFKGWQPHFYGTGLDNSTVGFLGMGAIGLAMADRLQGWGATLQYHAAKALDTQTEQRLGLRQVACSELFASSDFILLALPLNADTLHLVNAELLALVRPGALLVNPCRGSVVDEAAVLAALERGQLGGYAADVFEMEDWARADRPLCIDPALLAHPNTLFTPHIGSAVRAVRLEIERCAAQNILQALAGERPINAVNRLPKANPAAD

SEQ ID NO: 6 - DNA, 1005 bp, synthetic construct
  source 1..1005: mol_type = other DNA
ctgccgaaactcgttataactcaccgagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttcaagggctggcaaccacatttctacggcacggggctggataactctacggtcggcttccttggcatgggcgccatcggactggccatggctgatcgcttgcagggatggggcgcgaccctgcagtaccacgcggcgaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatggaagactgggctcgcgcggaccggccgctgtgcatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccaatcctgccgcagac

(19) United States
(12) Patent Application Publication
(10) Pub. No.: US 2026/0337583 A1
(43) Pub. Date: Oct. 29, 2026

(54) CYTOCHROME P450 ENZYME, NUCLEIC ACID MOLECULE, AND GLYCYRRHETINIC ACID DERIVATIVE AND USE THEREOF

(71) Applicant: CHINA PHARMACEUTICAL UNIVERSITY, Jiangsu (CN)

(72) Inventors: Liaobin DONG, Jiangsu (CN); Xiaowei ZHANG, Jiangsu (CN); Xiaoxu LIN, Jiangsu (CN); Zengyuan WANG, Jiangsu (CN); Fangru LI, Jiangsu (CN)

(73) Assignee: CHINA PHARMACEUTICAL UNIVERSITY, Jiangsu (CN)

(21) Appl. No.: 19/464,519

(22) Filed: Jan. 29, 2026

Related U.S. Application Data
(63) Continuation of application No. PCT/CN2024/115381, filed on Aug. 29, 2024.

(30) Foreign Application Priority Data
Sep. 19, 2023 (CN) 202311209506.9

(51) Int. Cl. C12N 9/02; A61K 31/56; A61P 35/00; C12N 15/70; C12P 33/00

(52) U.S. Cl. CPC C12N 9/001; A61K 31/56; A61P 35/00; C12N 15/70; C12P 33/00; C12Y 103/0701

(57) ABSTRACT

Provided are a cytochrome P450 enzyme, a nucleic acid molecule, and a glycyrrhetinic acid derivative and a use thereof. By means of an in-vitro catalytic conversion system constructed by CYP161H12, selective C—H bond activation is achieved at a B ring and/or a D ring of a glycyrrhetinic acid skeleton molecule. C—H bond hydroxylation reaction achieved using the enzyme-catalysis technology has excellent site- and stereoselectivity, involves mild reaction conditions, is environmentally friendly, and conforms to the principle of green chemistry. An enzyme in-vitro catalytic conversion system constructed can generate a clean product profile, and facilitate the separation and purification of a product. A novel glycyrrhetinic acid derivative synthesized retains hydroxyl groups on the B ring and the D ring, which has important significance in expanding the chemical space of a glycyrrhetinic acid derivative and deeply exploring a structure-activity relationship of the glycyrrhetinic acid derivative in pharmacological research.

### CROSS-REFERENCE TO RELATED APPLICATION

This application is a continuation of international application of PCT application serial no. PCT/CN2024/115381, filed on Aug. 29, 2024, which claims the priority benefit of China application no. 202311209506.9 filed on Sep. 19, 2023. The entirety of each of the above mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Jan. 21, 2026, is named 163872-US-Sequence List and is 10,578 bytes in size.

### TECHNICAL FIELD

The present disclosure belongs to the technical field of genetic engineering and pharmaceuticals, and in particular to a cytochrome P450 enzyme, a nucleic acid molecule, and a glycyrrhetinic acid derivative and a use thereof, and specifically relates to a use of the same in the preparation of a novel glycyrrhetinic acid derivative.

### RELATED ART

Glycyrrhetinic acid is a common oleanane-type pentacyclic triterpenoid natural product. At present, a series of glycyrrhetinic acid derivatives obtained by structural modification of a glycyrrhetinic acid core exhibit broad pharmacological activities, including anti-inflammatory, antitumor, antiviral, and neuroprotective effects. Traditional organic synthesis methods mainly include two strategies: derivatization based on the hydroxyl group on a ring A, the conjugated enone on a ring C, and the C30 carboxyl group on a ring E of the glycyrrhetinic acid skeleton; and derivatization based on a directing group strategy to activate nearby C—H bonds, thereby improving its pharmacological activity. However, since the rings B and D of glycyrrhetinic acid predominantly contain chemically similar, inert sp³-hybridized C—H bonds and lack directing groups, selective C—H activation on the rings B/D of glycyrrhetinic acid cannot currently be achieved through organic synthesis methods.

Cytochrome P450 enzymes (CYP450s for short) are a class of metalloenzymes that use ferrous-porphyrin as a catalytic center, catalyze monooxygenation reaction of substrates using molecular oxygen as an oxidant. Moreover, a microenvironment formed by interactions between the substrates and different amino acid residues in the enzyme active pocket endows CYP450s with unique regioselectivity and stereoselectivity. For a natural product with complex structure and containing multiple inert C—H bonds with similar chemical environments, such as glycyrrhetinic acid, CYP450s are ideal catalysts for achieving selective C—H bond activation and hydroxylation.

Typically, CYP450s require a reducing protein to facilitate electron transfer and a sufficient supply of NADPH for monooxygenation. Specifically, the naturally fused P450RHF system possesses a highly efficient electron-transfer chain; its fused reductase P450RHFRed is highly compatible with many CYP450s. The resulting artificial fusion enzyme, CYP450-RHFRed, exhibits high catalytic efficiency and is easy to express and use. Opt13 is a phosphite dehydrogenase that efficiently converts NAD⁺ into the expensive cofactor NADPH using Na₂HPO₃ in a mild aqueous system, enabling cofactor recycling. It is widely used in the catalytic transformation of CYP450s.

Currently, the reported methods for selective C—H hydroxylation of glycyrrhetinic acid using enzymes mainly relies on whole-cell transformation, in which microorganisms such as Bacillus megaterium catalyze the reaction using their endogenous enzymes. However, these methods often produce complex product backgrounds, posing challenges for separation and purification. Although in vitro enzymatic catalysis provides cleaner product profiles and is more suitable for large-scale application, no bacterial CYP450 gene capable of catalyzing selective C—H hydroxylation of glycyrrhetinic acid has yet been identified or cloned.

### SUMMARY OF INVENTION

Objectives of the present disclosure: A technical problem to be solved by the present disclosure is to provide a novel cytochrome P450 enzyme or a catalytically active fragment thereof capable of catalyzing the selective C—H bond hydroxylation of glycyrrhetinic acid.

Another technical problem to be solved by the present disclosure further addresses the technical problem of providing a nucleic acid molecule encoding the protein, as well as an expression cassette, a recombinant vector, and a recombinant strain or cell.

Yet another technical problem to be solved by the present disclosure is to provide an enzyme-catalyzed conversion system with CYP161H12 as a key catalytic enzyme, and to apply the system to an in vitro biotransformation of various glycyrrhetinic acid derivatives.

Still another technical problem to be solved by the present disclosure is to provide a use of the cytochrome P450 enzyme or the catalytically active fragment thereof, the nucleic acid molecule, the expression cassette, the recombinant vector, and the recombinant strain or the cell, the recombinant vector, the recombinant strain, and the enzyme-catalyzed conversion system in the preparation of a glycyrrhetinic acid derivative.

Still another technical problem to be solved by the present disclosure is to provide a series of novel glycyrrhetinic acid derivatives.

Still another technical problem to be solved by the present disclosure is to provide a method for preparing the glycyrrhetinic acid derivative by combining organic synthesis and enzyme catalysis.

A final technical problem to be solved by the present disclosure is to provide a use of the glycyrrhetinic acid derivative in the preparation of anti-inflammatory, antitumor, antiviral, or neuroprotective medicaments.

Technical solutions: in order to solve the above technical problems, the present disclosure provides a cytochrome P450 enzyme or a catalytically active fragment thereof, including:
(a) an amino acid sequence thereof is shown in SEQ ID NO: 2; or
(b) the amino acid sequence of the item (a) having been substituted and/or deleted and/or added one or more amino acids, and having cytochrome P450 enzymatic activity.

The present disclosure further provides a nucleic acid molecule encoding the cytochrome P450 enzyme or the catalytically active fragment thereof.

Specifically, the nucleic acid molecule includes:
(a) the nucleic acid molecule encoding the cytochrome P450 enzyme or the catalytically active fragment thereof shares at least 70% sequence identity with SEQ ID NO: 2; and
(b) a nucleotide sequence thereof is shown in SEQ ID NO: 1.

A cytochrome oxidase gene of the present disclosure belongs to A CYP161H subfamily and is designated CYP161H12 according to the CY P450 international nomenclature. An amino acid sequence (404 aa) of the protein encoded by CYP161H12 is shown in SEQ ID NO: 1 of the Sequence Listing, and a nucleotide sequence (1212 bp) of the same is shown in SEQ ID NO: 2.

The CYP161H1 protein sequence is identified and selected through a survey and screening of the NCBI database. CYP161H12 is derived from Amycolatopsis pretoriensis, with an amino acid sequence UniProt No. A0A1H5RFX8. Codon bias optimization is performed based on the protein sequence, and an optimized sequence is shown in SEQ ID NO: 2. The gene encoding the enzyme is then directly synthesized by General Biol (Anhui) Co., Ltd. and cloned into a pET28a-RHFRed vector, with restriction endonuclease sites of NdeI and BamHI, respectively, thereby generating a recombinant plasmid pET28a-CYP161H12-RHFRed.

The present disclosure further includes an expression cassette, a recombinant vector, and a recombinant strain or cell including the nucleic acid molecule.

The present disclosure further includes a recombinant vector, where the recombinant vector is obtained by introducing the nucleic acid molecule into the expression vector. Preferably, the expression vector is the plasmid pET28a-RHFRed.

The present disclosure also provides a recombinant strain, where the recombinant strain is obtained by introducing the recombinant vector into Escherichia coli (E. coli). Preferably, the E. coli includes E. coli BL21.

The present disclosure further provides an enzyme-catalyzed conversion system, including a cell lysate 1 from the recombinant strain. Preferably, the enzyme-catalyzed conversion system further includes a cell lysate 2 from a recombinant strain expressing phosphite dehydrogenase. Preferably, a volume ratio of the cell lysate 1 to the cell lysate 2 is 2:1.

The present disclosure further includes a use of the cytochrome P450 enzyme or the catalytically active fragment thereof, the nucleic acid molecule, the expression cassette, the recombinant vector, and the recombinant strain or the cell, the recombinant vector, the recombinant strain, and the enzyme-catalyzed conversion system in the preparation of a glycyrrhetinic acid derivative.

The present disclosure further provides a glycyrrhetinic acid derivative having a following structural formula:

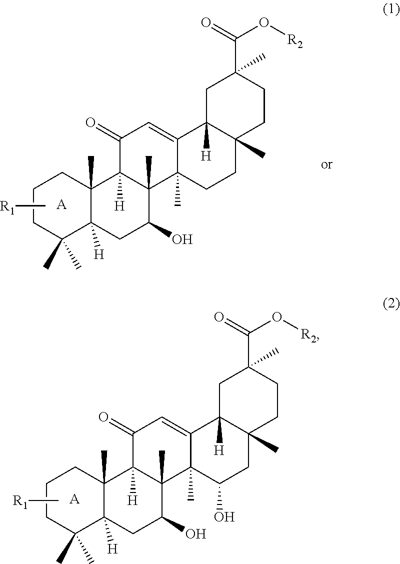

where R1 is a functional group on a ring A of glycyrrhetinic acid or a derivative structure based on modifications of the ring A. The functional group on the ring A includes: a hydroxyl group at C3, an acetyl group at C3, a propionyl group at C3, a butyryl group at C3, and a ketone carbonyl group at C3. The derivative structure based on modifications of the ring A includes an α,β-unsaturated enone of C3-oxo-C1-ene, a seven-membered lactone ring formed by ring expansion of the ring A, and fused isoxazole heterocycles at C2 and C3, where R2 includes H, methyl or hexenyl.

Specifically, the glycyrrhetinic acid derivative has the following structural formulas:

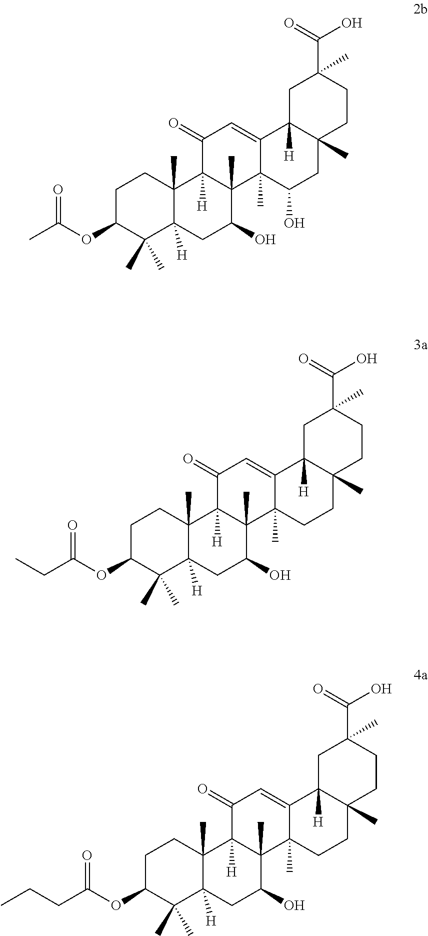

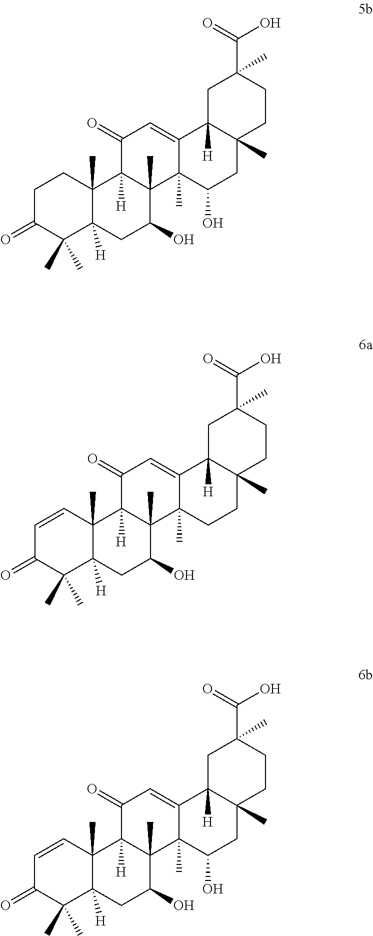

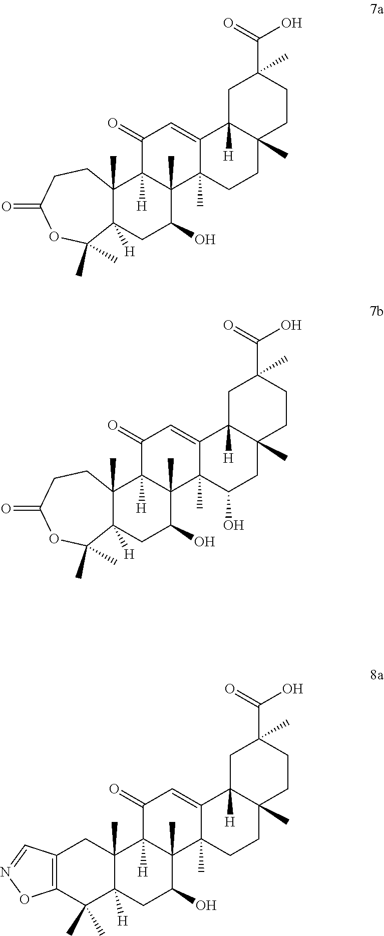

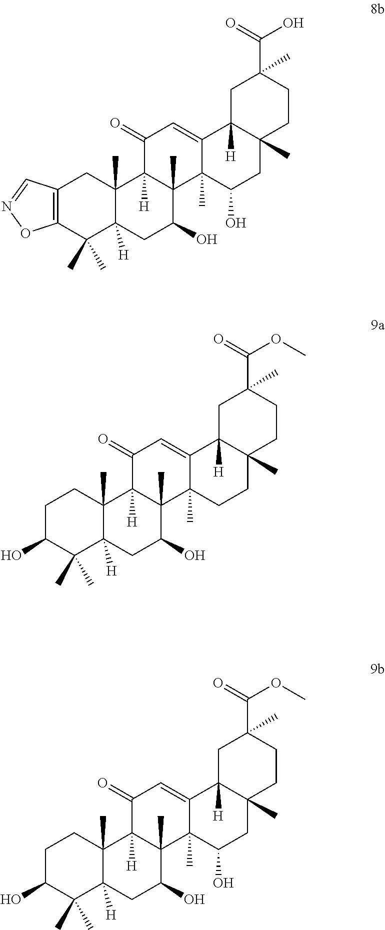

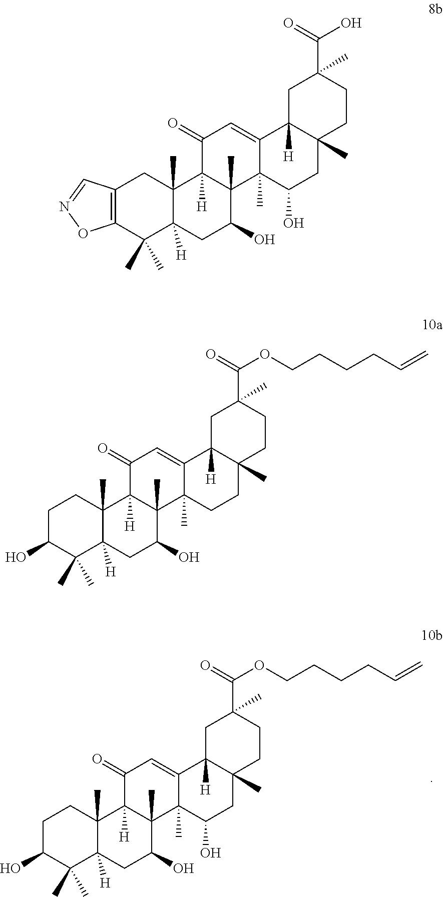

The present disclosure further provides a method for preparing the glycyrrhetinic acid derivative by combining organic synthesis and enzyme catalysis, including the following steps:
1) constructing a recombinant expression vector: the nucleic acid or gene is introduced into an expression vector to obtain the recombinant expression vector;
2) preparing and fermenting a recombinant strain: the recombinant expression vector is introduced into a host strain to obtain a recombinant strain; fermenting and the recombinant strain is fermented and induced to express, followed by ultrasonic disruption to obtain a cell lysate 1;

preferably, the method further includes fermenting and inducing expression of a recombinant strain expressing phosphite dehydrogenase, followed by ultrasonic disruption to obtain a cell lysate 2; and
3) preparing a glycyrrhetinic acid derivative by catalysing a substrate: the cell lysate 1 is reacted with the substrate to obtain the glycyrrhetinic acid derivative; preferably, it further includes adding the cell lysate 2 for reaction; preferably, the substrate includes glycyrrhetinic acid, 3-hydroxy-glycyrrhetinic acid, 3-acetyl-glycyrrhetinic acid, 3-propionyl-glycyrrhetinic acid, 3-butyryl-glycyrrhetinic acid, 3-oxo-glycyrrhetinic acid, 3-oxo-1-ene-glycyrrhetinic acid, and glycyrrhetinic acid derivatives with a seven-membered lactone ring on a ring A.

Preferably, the recombinant expression vector in the step (1) includes pET28a-CYP161H12-RHFRed.

Preferably, the fermentation steps in the step (2) includes: inoculating a recombinant E. coli containing pET28a-CYP161H12-RHFRed into a lysogeny broth (LB) medium, and culturing at 37° C. to obtain a seed culture; adding the seed culture and a trace metal salt solution to the TB medium; culturing at 37° C. until a logarithmic growth phase occurs; then adding IPTG and 5-ALA to induce bacterial expression of the CYP161H12-RHFRed fusion protein, followed by induction at 18° C. for 20 h; and finally collecting the cells by centrifugation for in vitro catalytic reactions.

Preferably, a formulation of the trace metal salt solution is: 50 mM FeCl₃, 20 mM CaCl₂, 10 mM MnSO₄, 10 mM ZnSO₄, 2 mM CoSO₄, 2 mM CuCl₂, 2 mM NiCl₂, 2 mM Na₂MoO₄, and 2 mM H₃BO₃; and preferably, the trace metal salt solution is added to the TB medium at a ratio of 0.1% v/v.

Preferably, a concentration of the IPTG in the TB medium is 0.1 mM, and a concentration of the 5-ALA in the TB medium is 0.5 mM.

The present disclosure further includes a method for constructing a multi-enzyme catalytic conversion system with CYP161H12 as a key catalytic enzyme, as well as a method for further preparing glycyrrhetinic acid derivatives, specifically including the following steps:
(1) heterologous expression of CYP161H12-RHFRed in E. coli: the recombinant E. coli containing pET28a-CYP161H12-RHFRed is inoculated into an LB medium and cultured at 37° C. to obtain a seed culture; the seed culture and a trace metal salt solution are then added to the TB medium, and then first incubated at 37° C. to a logarithmic growth phase; isopropyl-β-D-thiogalactopyranoside (IPTG) and 5-aminolevulinic acid hydrochloride (5-ALA) are then added to induce bacterial expression of the CYP161H12-RHFRed fusion protein; induction is carried out at 18° C. for 20 h; and cells are collected by centrifugation for in vitro catalytic reactions. The implementation method is specified in Example 1 below.
(2) Heterologous expression of opt13 in E. coli: the opt13 gene is ligated into the pETDuet vector using seamless cloning to obtain a recombinant plasmid pETDuet-opt13, which is then transformed into E. coli BL21; the recombinant E. coli is inoculated into an LB medium and cultured at 37° C. to obtain a seed culture; the seed culture is added to the TB medium and first cultured at 37° C. to a logarithmic growth phase, IPTG is then added to induce bacterial expression of the opt13; IPTG is then added to induce bacterial expression of the opt13; induction is carried out at 18° C. for 20 h; and cells are collected by centrifugation for in vitro catalytic reactions. The implementation method is specified in Example 2 below.
(3) In vitro catalytic conversion reaction: the fermentation cells obtained from the steps (1) and (2) are resuspended in a KPi buffer and sonicated to obtain cell lysates of CYP161H12-RhfRed and opt13, respectively; the two lysates are then mixed at a volume ratio of 2:1 to generate a three-component in vitro catalytic system containing the cytochrome P450 enzyme CYP161H12, the reductase RHFRed, and the phosphite dehydrogenase opt13 for efficient NADPH regeneration. Substrates, Na₂HPO₃·5H₂O and NADP⁺, are added to the three-component in vitro catalytic system, and the reaction is conducted at 23° C. for 20 h to obtain a reaction product; the reaction product is then extracted, concentrated, and separated to obtain the oxidized products with inert C—H bond hydroxylation on the ring B/D.

In the step (3), the KPi buffer solution is prepared by dissolving 10.27 g of K₂HPO₄·3H₂O and 0.68 g of KH₂PO₄ in 1 L of distilled water to obtain a phosphate buffer with pH=7 and a concentration of 0.1 M.

The concentration of the KPi buffer mentioned in the step (3) is preferably 0.1 M, and the pH is preferably 7.0.

The novel glycyrrhetinic acid derivative of the present disclosure may first be synthesized through organic chemical synthesis to obtain ring-A glycyrrhetinic acid derivative, followed by CYP161H12 catalysis to obtain a glycyrrhetinic acid derivative with ring A derivatization and C—H bond hydroxylation on rings B and/or D, as shown in a reaction route 1.

The reaction route 1 is shown as follows:

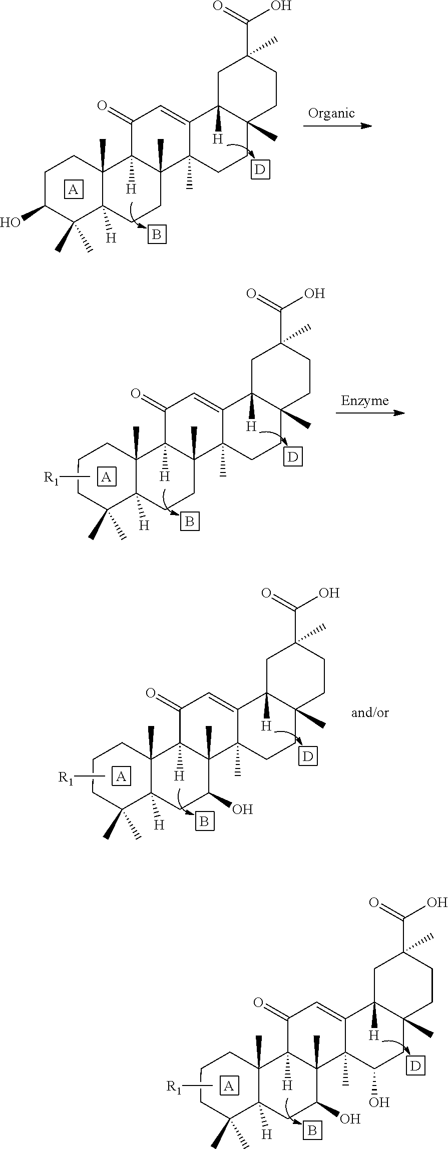

In the reaction route 1, the derivatives obtained after modifying the ring A of glycyrrhetinic acid exhibit different catalytic oxidation efficiencies compared with CYP161H12. For some derivatives, products with hydroxylation are only generated at a C7β position, as shown in Examples 8 and 9. For other derivatives, dihydroxy products at C7β and C15α may be obtained through an oxidative cascade, as shown in Examples 7, 10, 11, 12, and 13.

The novel glycyrrhetinic acid derivatives of the present disclosure may also be obtained by first hydroxylating the ring B at C7β and/or ring D at C15α of glycyrrhetinic acid through CYP161H12 catalysis, followed by organic synthesis to esterify a C30 carboxyl group on the glycyrrhetinic acid skeleton, which yields a product of with C30 carboxyl esterification and C—H bond hydroxylation on rings B and/or D, as shown in a reaction route 2.

The reaction route 2 is shown as follows:

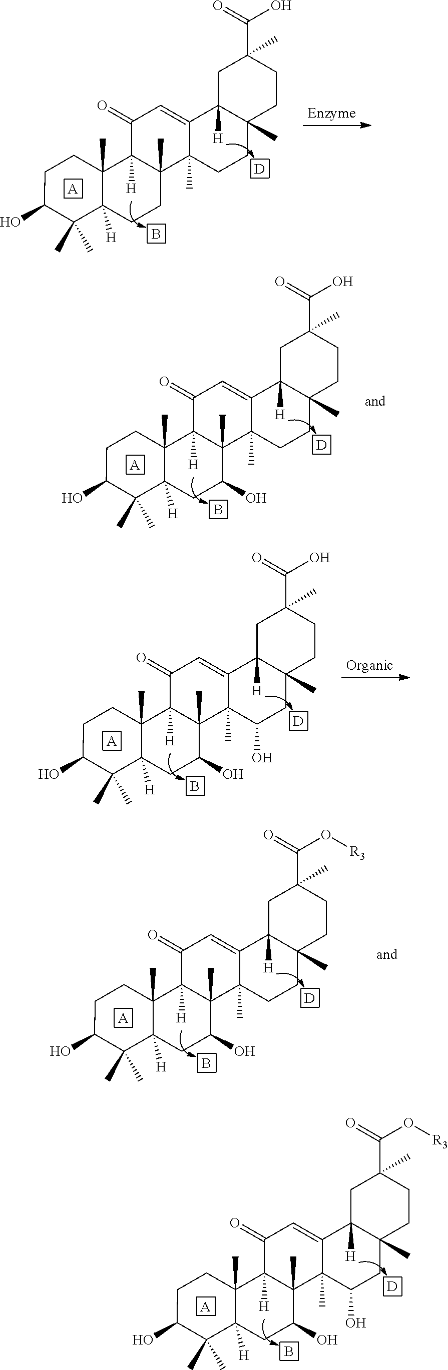

In Reaction Scheme 2, CYP161H12 may be subjected to an oxidative cascade at C7β and C15α of glycyrrhetinic acid, generating two hydroxylated products. Subsequent esterification of the carboxyl group can be readily carried out without affecting the existing hydroxyl groups.

The present disclosure further provides a use of the glycyrrhetinic acid derivatives in the preparation of anti-inflammatory, antitumor, antiviral, or neuroprotective medicaments.

Beneficial effects: Compared with the prior art, the present disclosure has the following advantages:
(1) The present disclosure provides a novel cytochrome enzyme, CYP161H12, and an encoding gene thereof. By means of an in-vitro catalytic conversion system constructed by CYP161H12, highly selective C—H bond activation is achieved at a B ring and/or a D ring of a glycyrrhetinic acid skeleton molecule. (2) Compared with traditional organic synthesis, the C—H bond hydroxylation achieved by enzyme catalysis of the present disclosure has significant advantages. The technology has excellent site- and stereoselectivity, involves mild reaction conditions, is environmentally friendly, and conforms to the principle of green chemistry. (3) Compared with the whole-cell transformation by microorganisms, the enzyme in-vitro catalytic conversion system constructed can generate a clean product profile, and facilitate the separation and purification of a product. (4) Hydroxyl groups, as common molecular handles, are easily modified to introduce other functional groups. The novel glycyrrhetinic acid derivatives synthesized in present disclosure retain hydroxyl groups at the rings B and/or D, which has important significance in expanding the chemical space of a glycyrrhetinic acid derivative and deeply exploring a structure-activity relationship of the glycyrrhetinic acid derivative in pharmacological research.

### BRIEF DESCRIPTION OF DRAWINGS

FIG. 1 is a schematic diagram of an in vitro catalytic conversion system of glycyrrhetinic acid using CYP161H12 as a key catalyst.

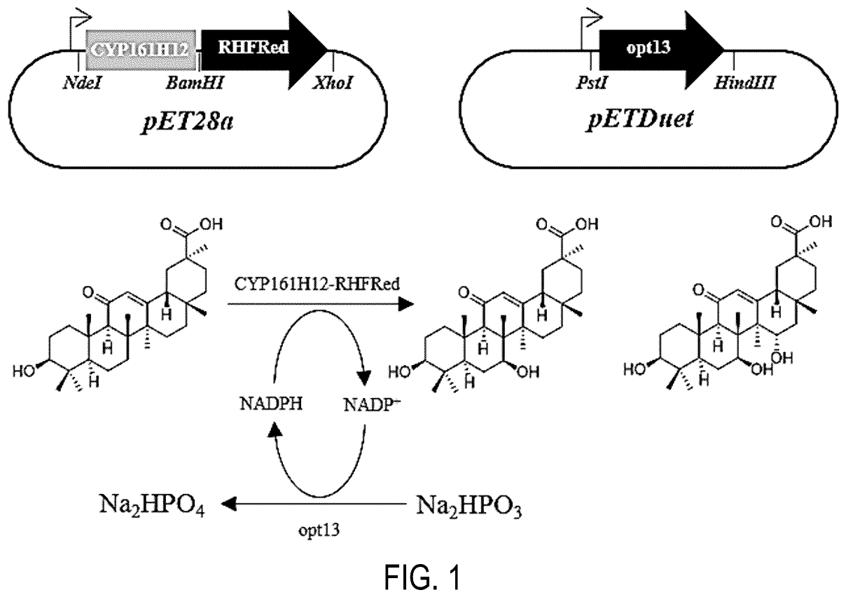

FIG. 2 is an ¹H NMR spectrum of Compound 2b.

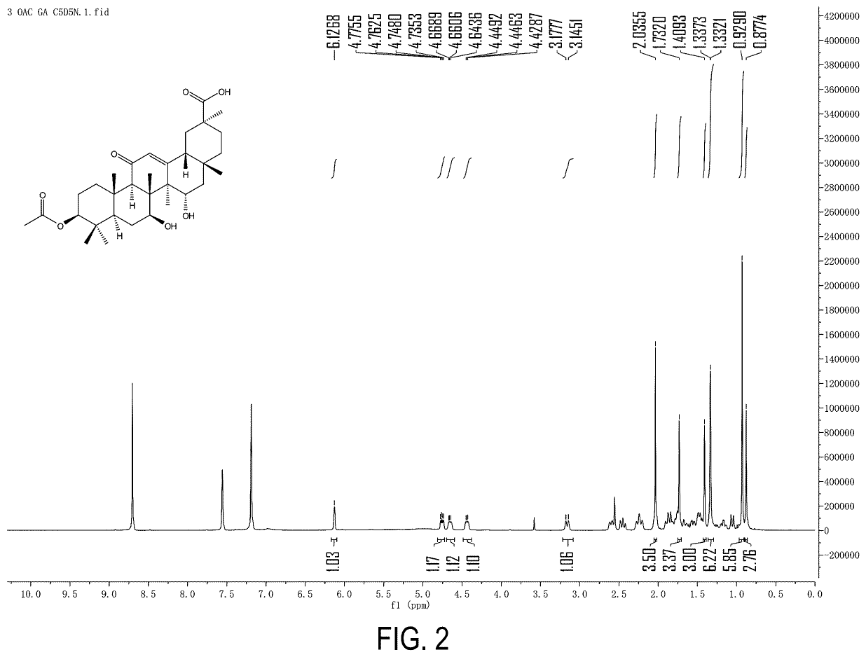

FIG. 3 is a ¹³C NMR spectrum of Compound 2b.

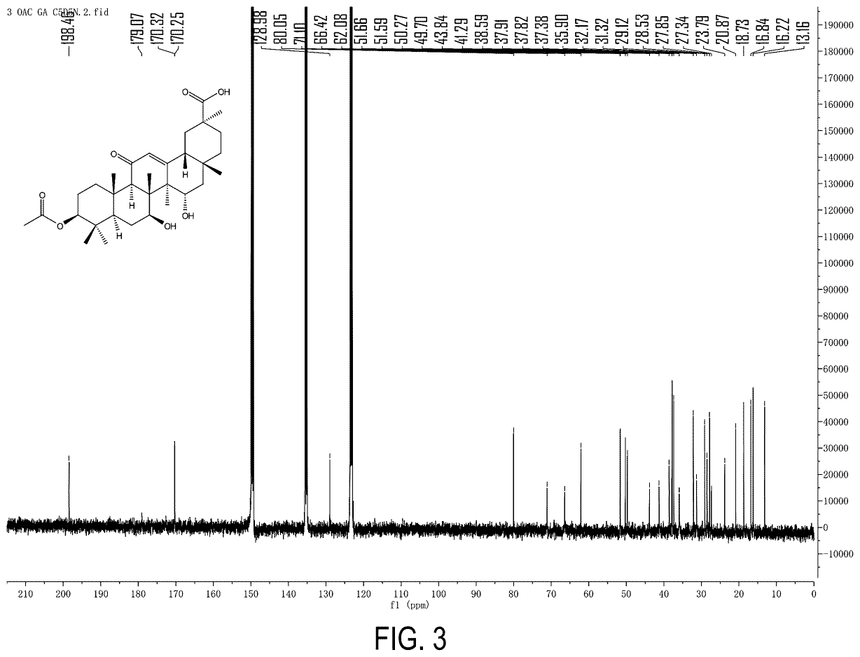

FIG. 4 is an ¹H NMR spectrum of Compound 3a.

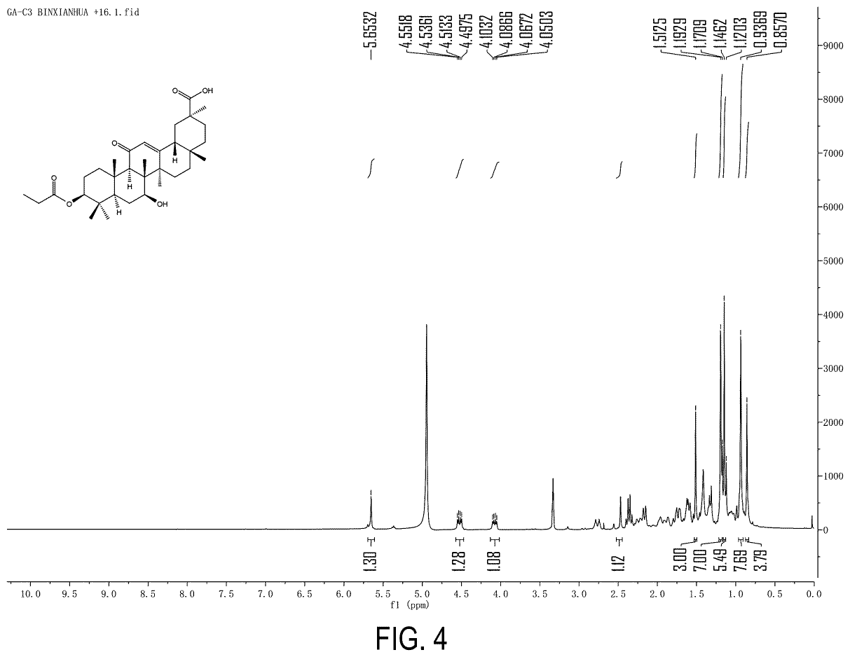

FIG. 5 is a ¹³C NMR spectrum of Compound 3a.

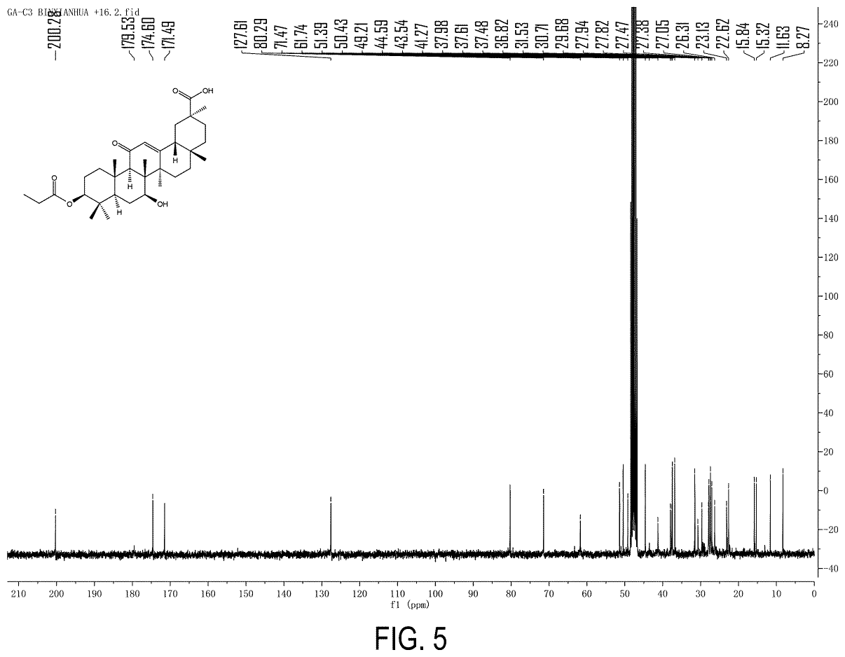

FIG. 6 is an ¹H NMR spectrum of Compound 4a.

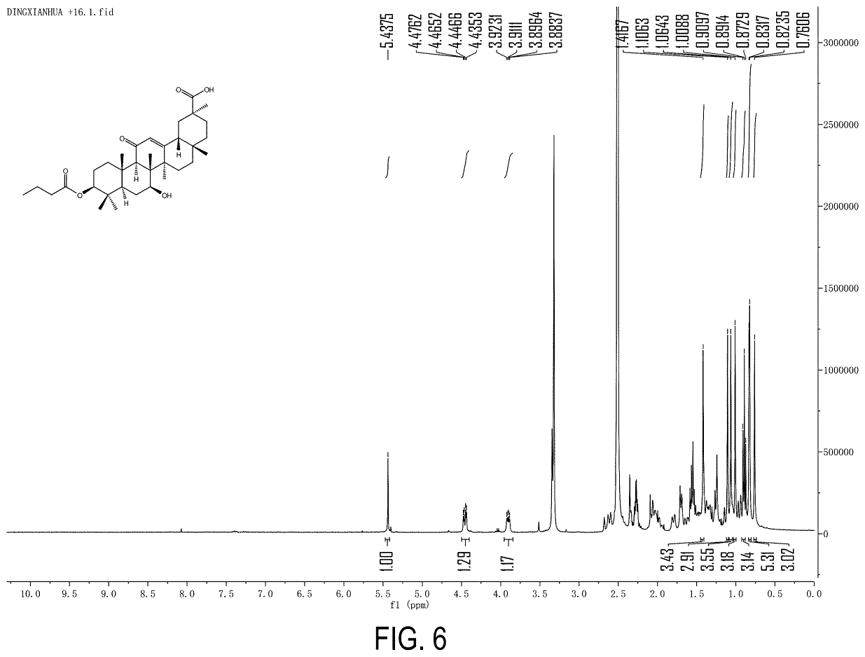

FIG. 7 is a ¹³C NMR spectrum of Compound 4a.

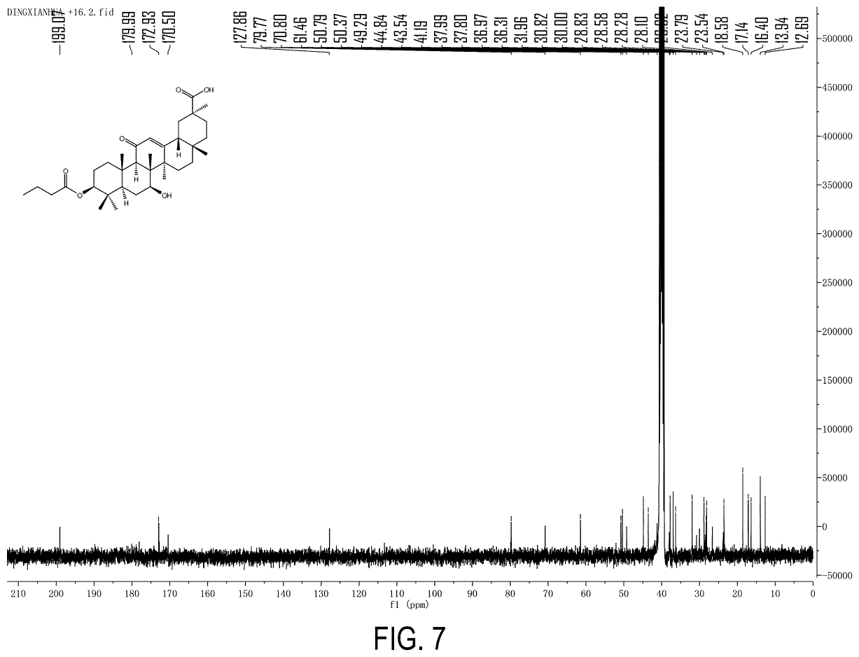

FIG. 8 is an ¹H NMR spectrum of Compound 5b.

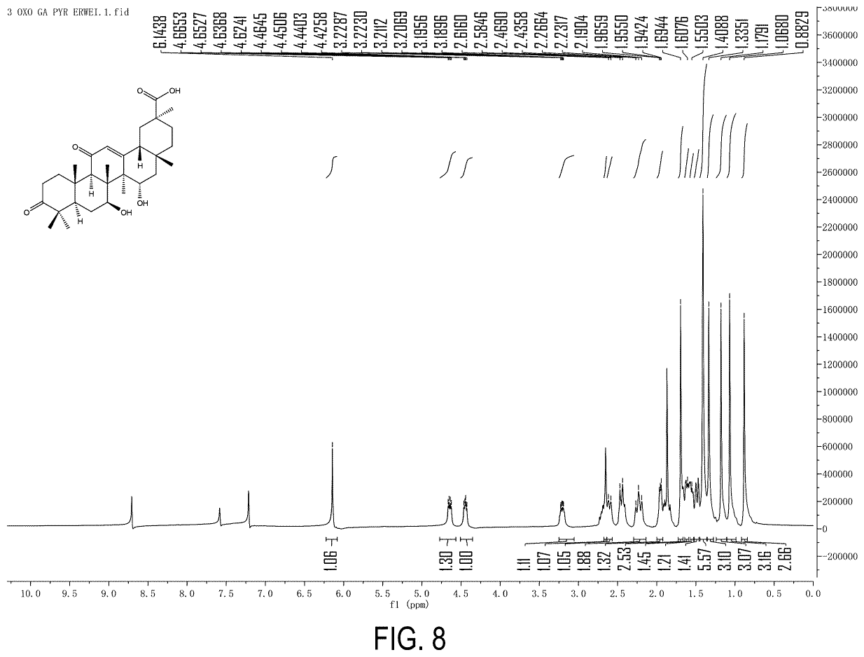

FIG. 9 is a ¹³C NMR spectrum of Compound 5b.

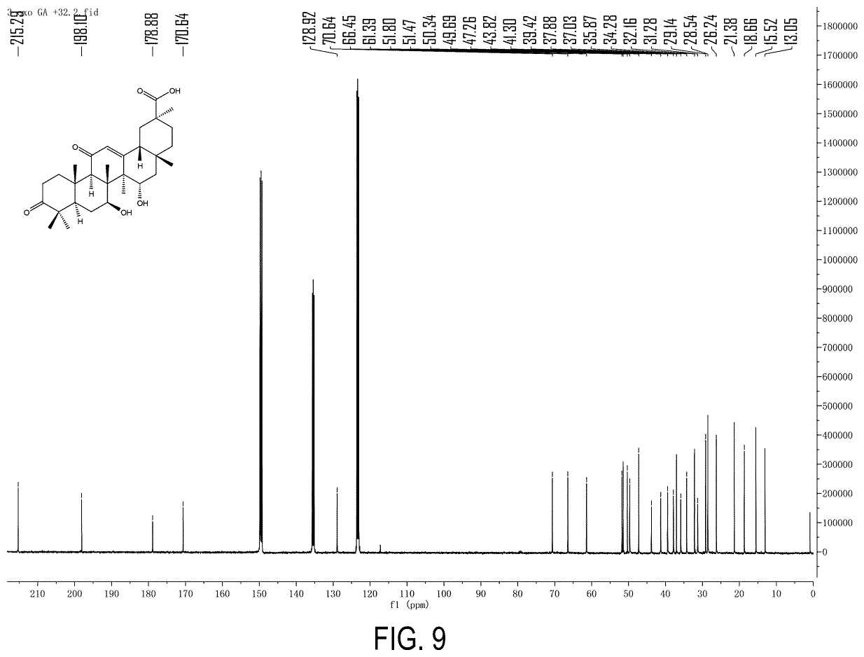

FIG. 10 is an ¹H NMR spectrum of Compound 6a.

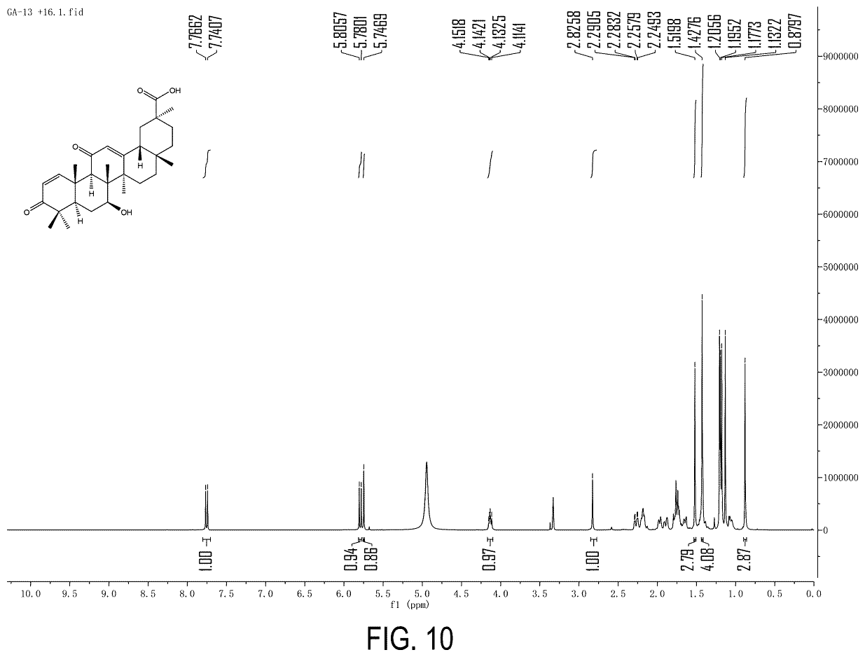

FIG. 11 is a ¹³C NMR spectrum of Compound 6a.

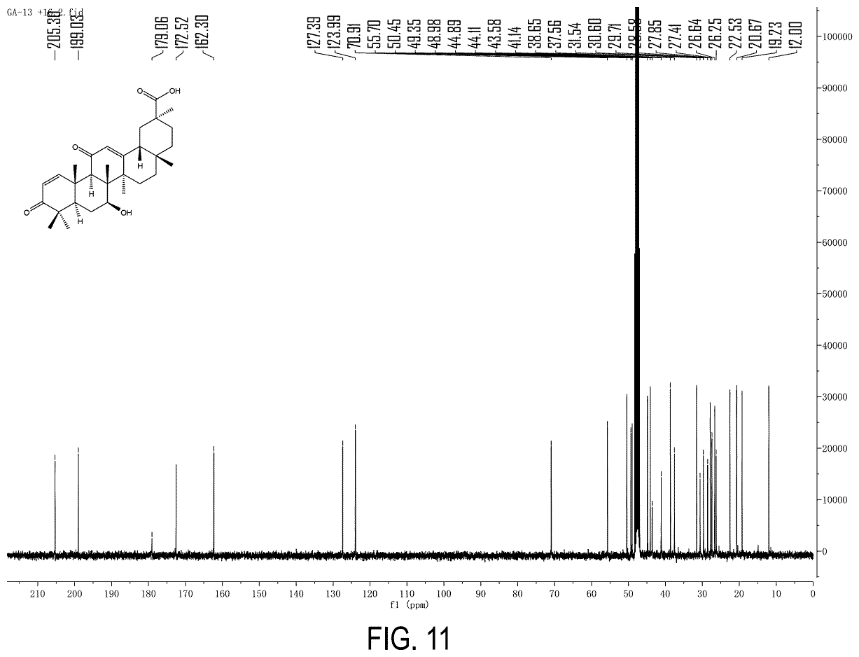

FIG. 12 is an ¹H NMR spectrum of Compound 6b.

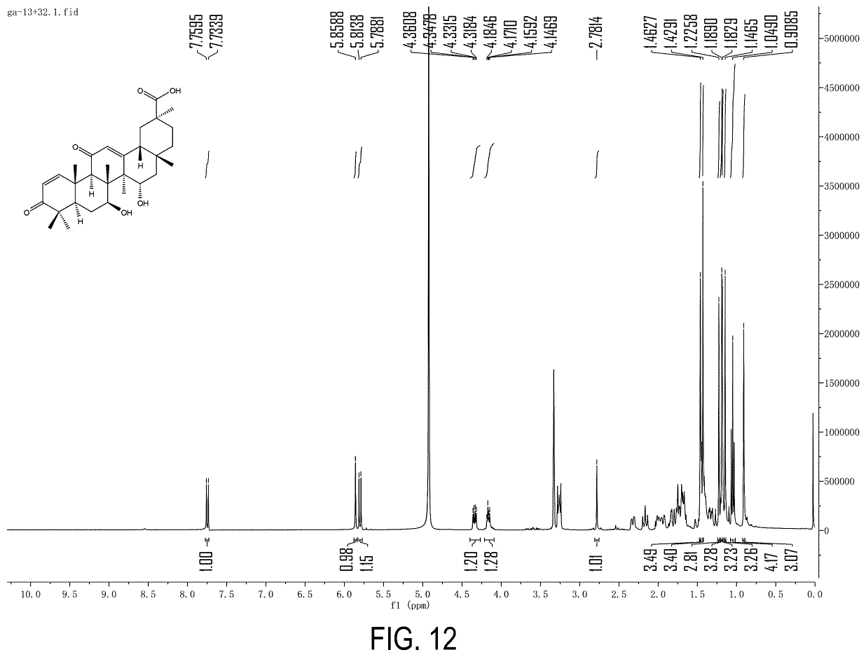

FIG. 13 is a ¹³C NMR spectrum of Compound 6b.

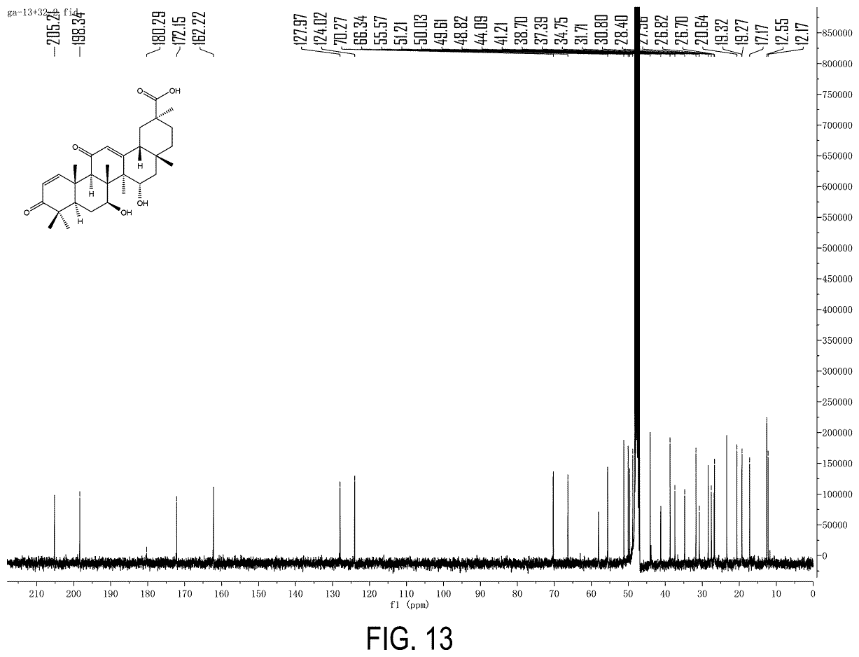

FIG. 14 is an ¹H NMR spectrum of Compound 7a.

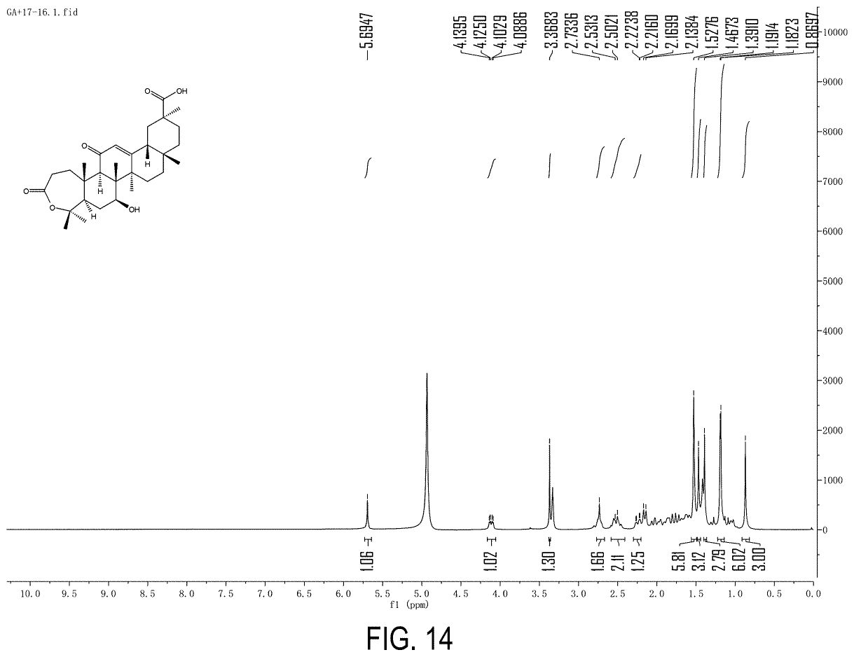

FIG. 15 is a ¹³C NMR spectrum of Compound 7a.

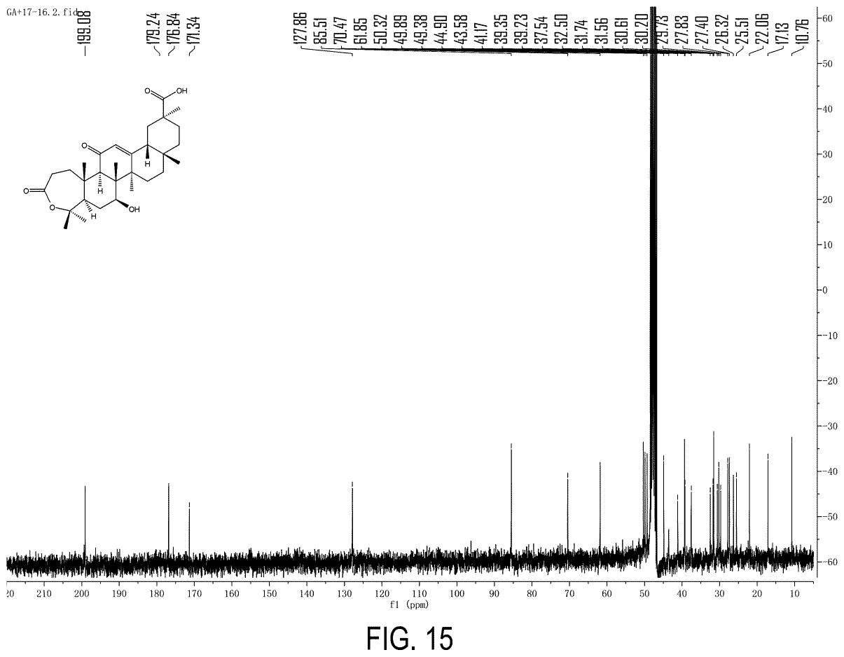

FIG. 16 is an ¹H NMR spectrum of Compound 7b.

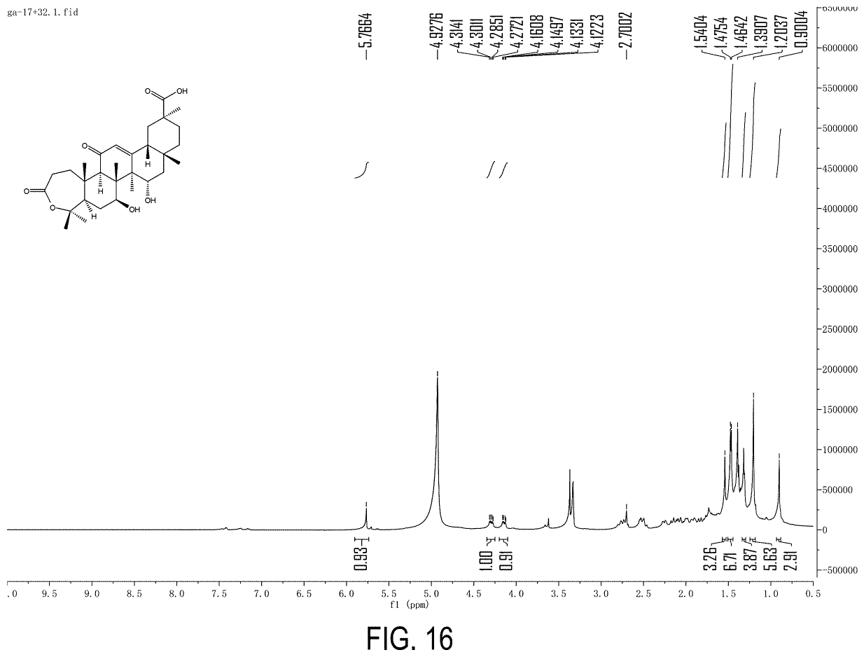

FIG. 17 is a ¹³C NMR spectrum of Compound 7b.

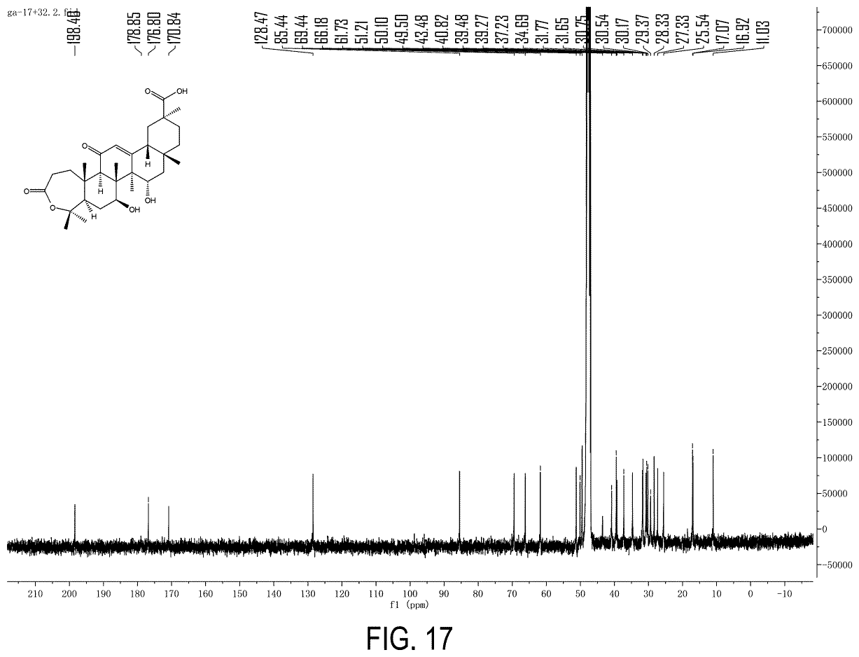

FIG. 18 is an ¹H NMR spectrum of Compound 8a.

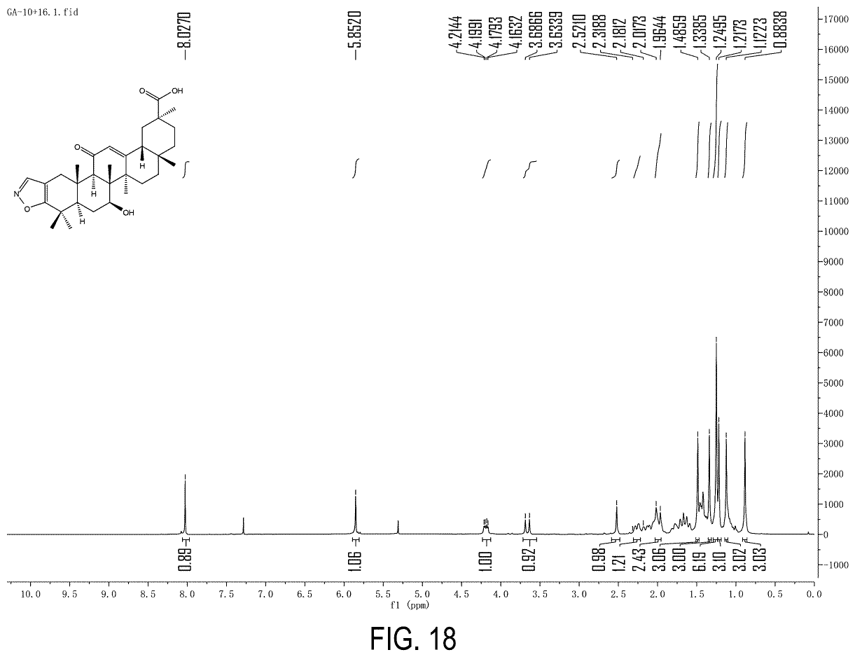

FIG. 19 is a ¹³C NMR spectrum of Compound 8a.

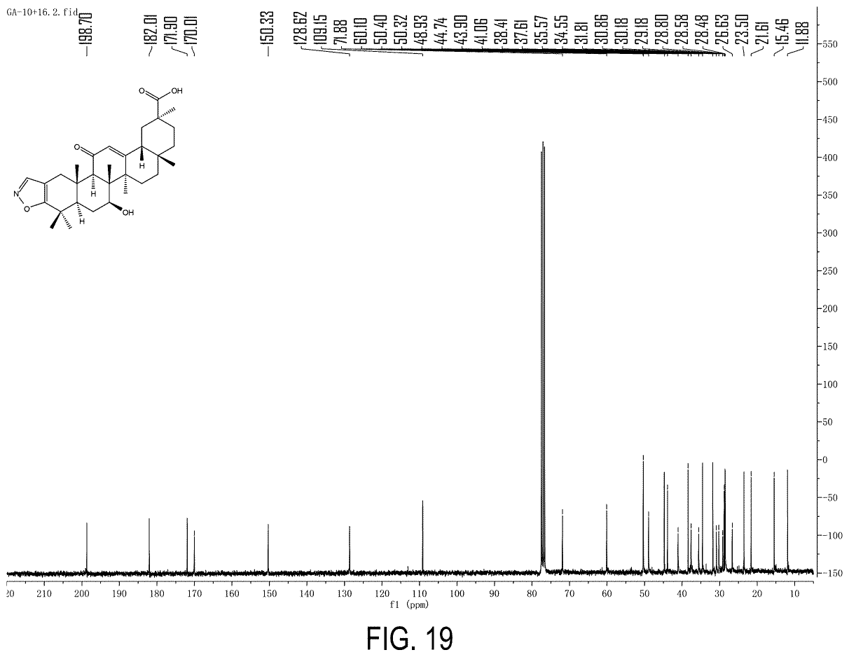

FIG. 20 is an ¹H NMR spectrum of Compound 8b.

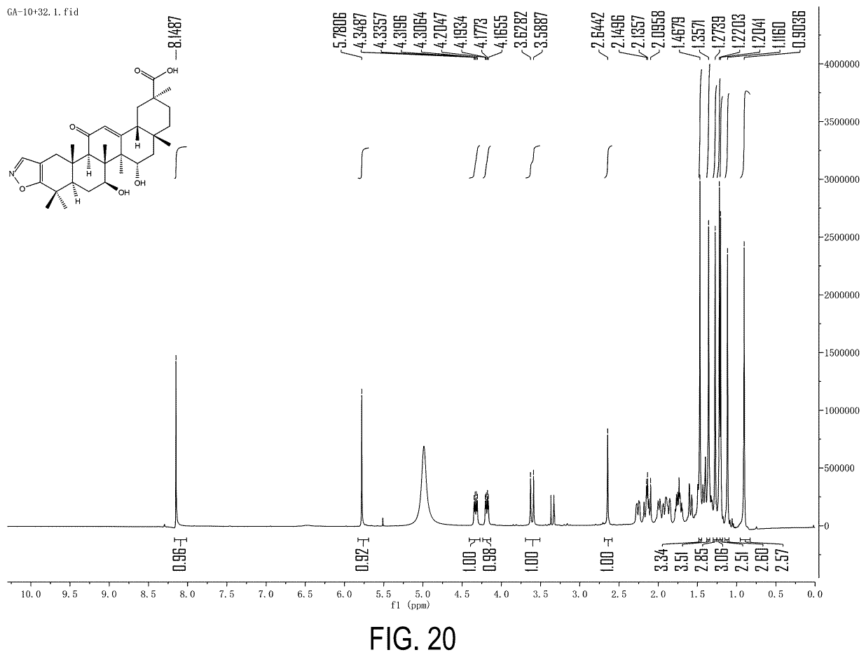

FIG. 21 is a ¹³C NMR spectrum of Compound 8b.

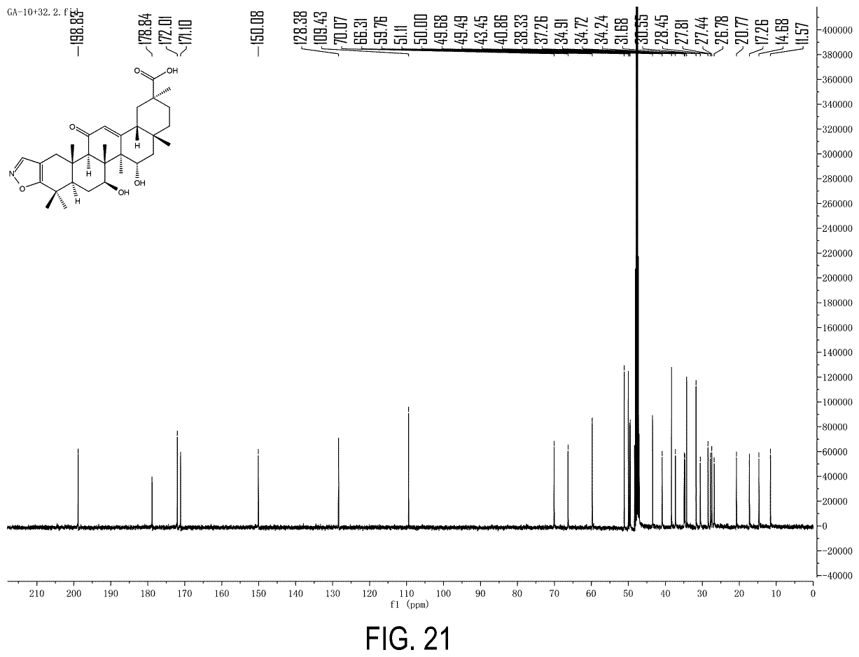

FIG. 22 is an ¹H NMR spectrum of Compound 9a.

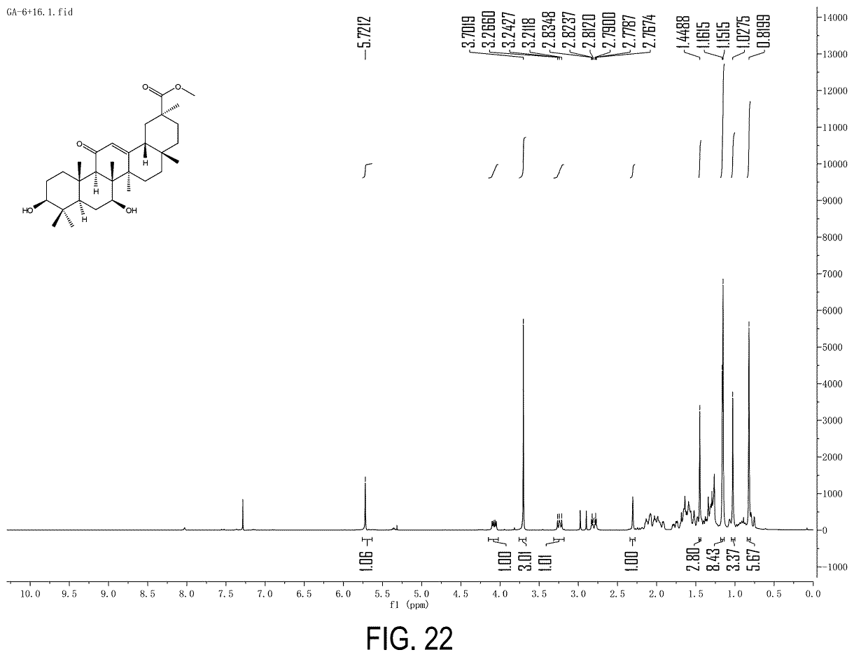

FIG. 23 is a ¹³C NMR spectrum of Compound 9a.

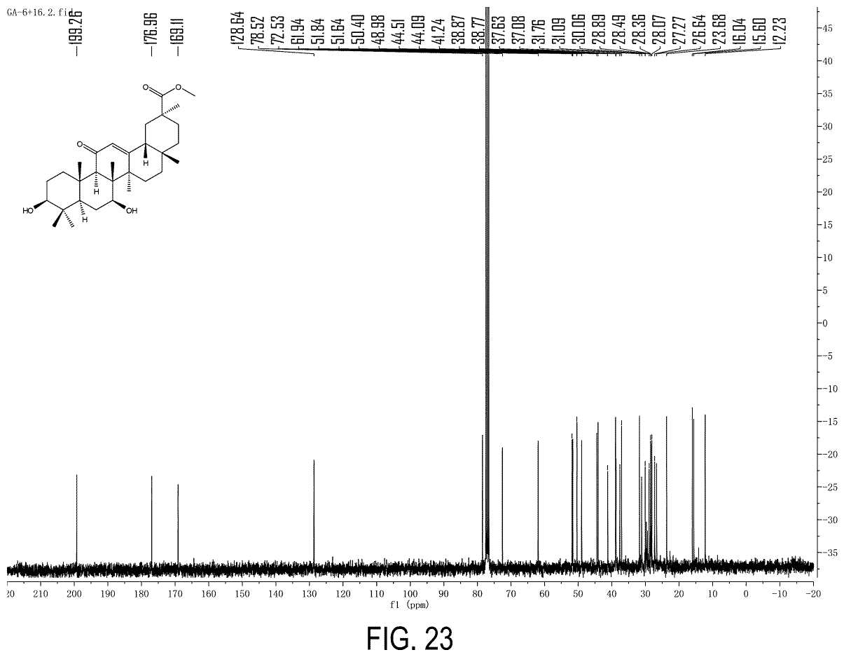

FIG. 24 is an ¹H NMR spectrum of Compound 9b.

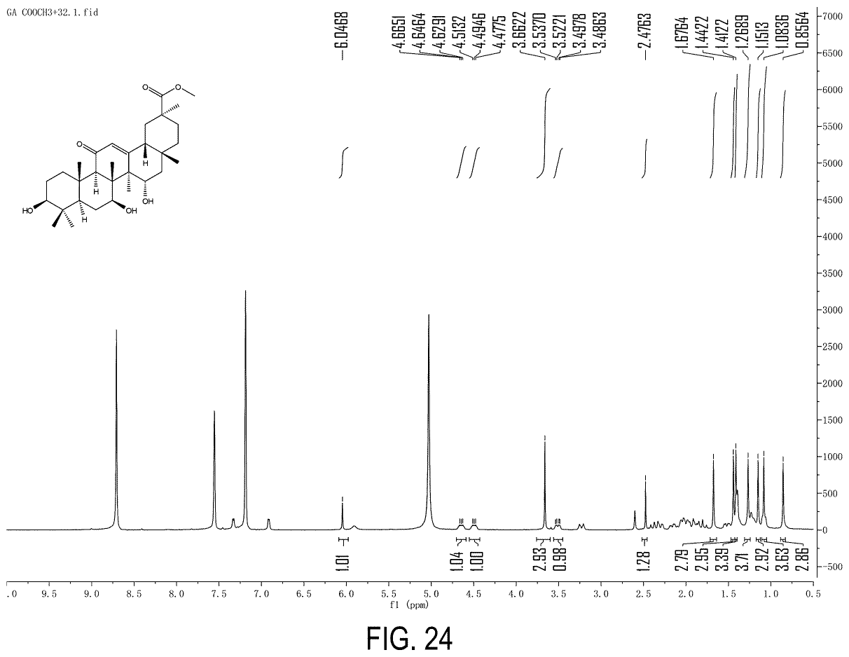

FIG. 25 is a ¹³C NMR spectrum of Compound 9b.

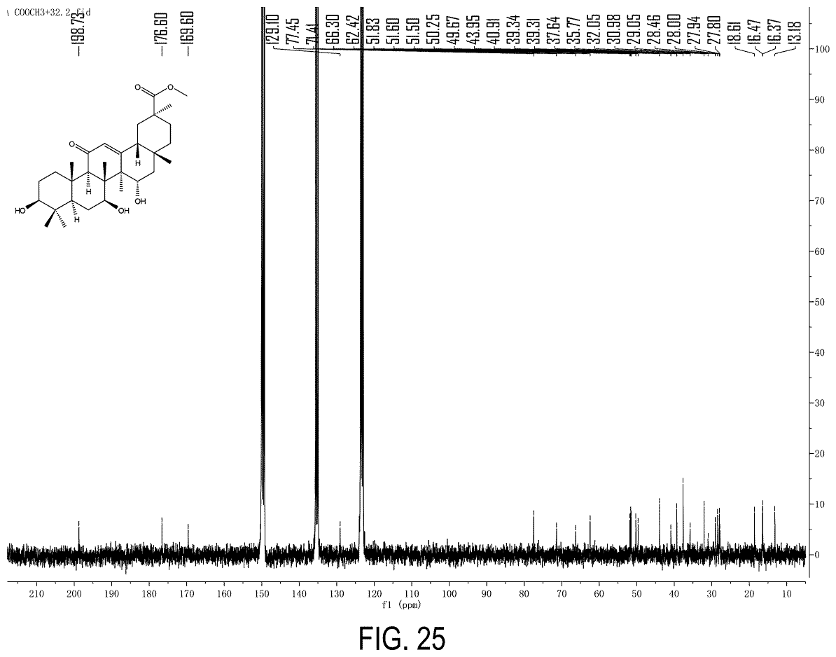

FIG. 26 is an ¹H NMR spectrum of Compound 10a.

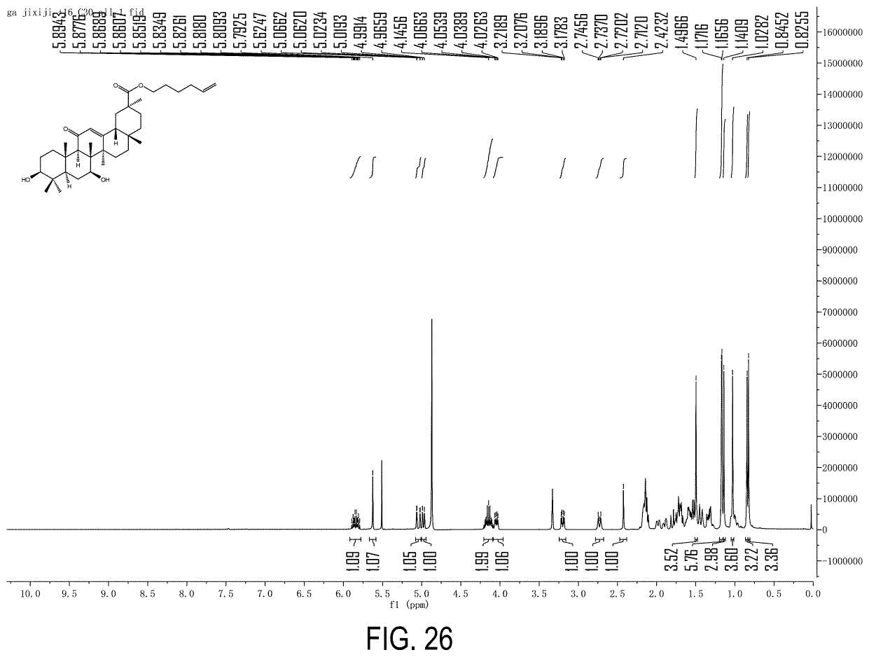

FIG. 27 is a ¹³C NMR spectrum of Compound 10a.

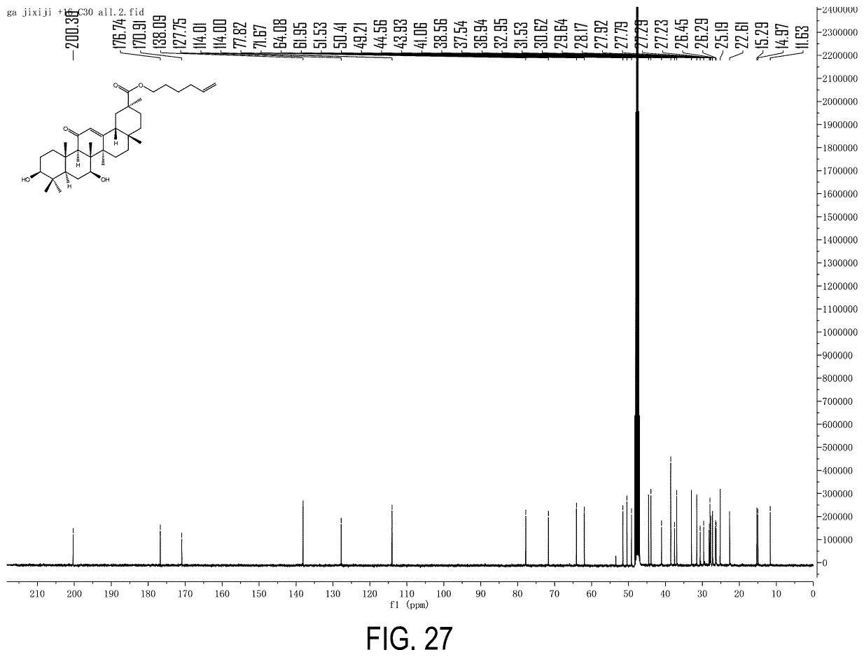

FIG. 28 is an ¹H NMR spectrum of Compound 10b.

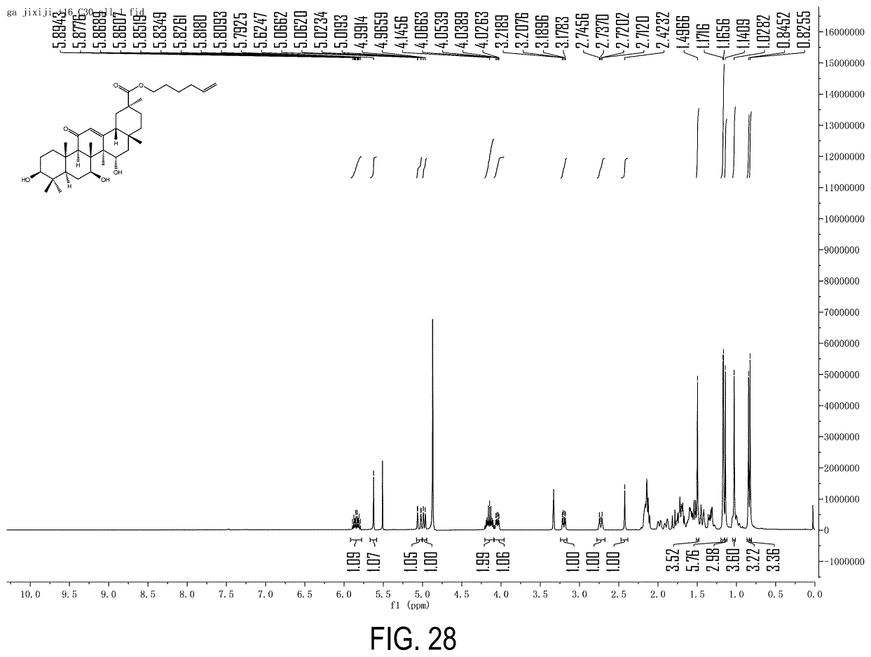

FIG. 29 is a ¹³C NMR spectrum of Compound 10b.

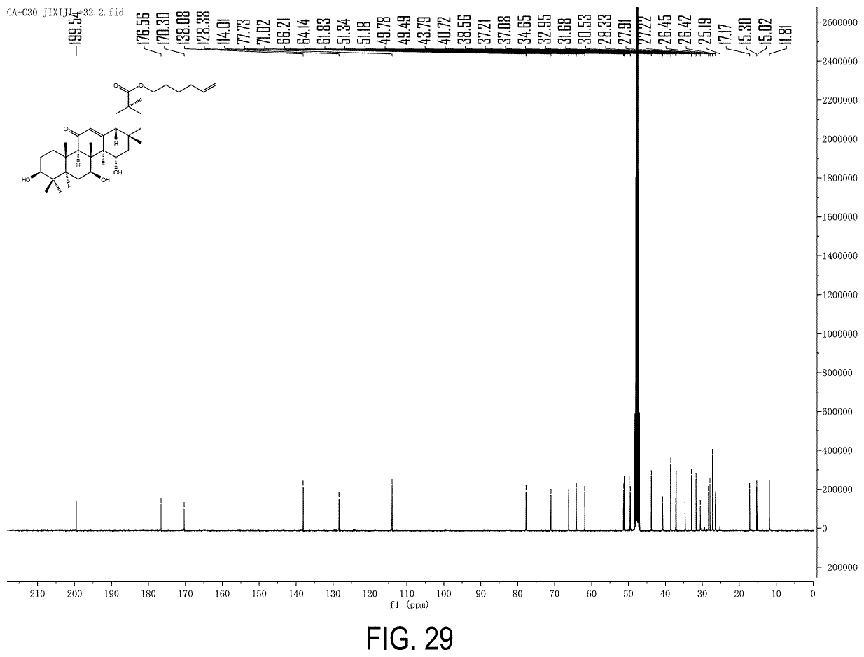

### DESCRIPTION OF EMBODIMENTS

Embodiments of the present disclosure will be described in detail below with reference to the examples, but those skilled in the art will understand that the following examples are for illustrative purposes only, and should not be considered as limiting the scope of the present disclosure. Any example without indicating specific conditions should be performed according to conventional conditions or conditions recommended by corresponding manufacturer. Reagents or instruments without indicating manufacturers are conventional products commercially available.

### Example 1 Heterologous Expression and Fermentation of CYP161H12-RHFRed in Escherichia coli (E. coli)

1. Construction of a Recombinant Expression Vector pET28a-CYP161H12-RHFRed

CYP161H12 was derived from Amycolatopsis pretoriensis, with an amino acid sequence UniProt No. A0A1H5RFX8. The sequence of CYP161H12 was subjected to codon optimization to match codon usage preference of E. coli, resulting in SEQ ID NO:2. Taking a pET28a-RHFRed plasmid as an expression vector, the nucleotide sequence of CYP161H12 (as shown in SEQ ID NO: 2) was inserted into the pET28a-RHFRed plasmid with restriction endonuclease sites of NdeI and BamHI, respectively. The recombinant vector pET28a-CYP161H12-RHFRed was directly synthesized by General Biol (Anhui) Co., Ltd.

Specifically, a synthesis process of the pET28a-RHFRed is as follows: based on an amino acid sequence of RHFRed reported in 2020, a RHFRed was inserted into the pET28a with restriction endonuclease sites of BamHI and XhoI, respectively. It was synthesized by General Biol (Anhui) Co., Ltd. to obtain the vector pET28a-RHFRed. A sequence of the pET28a-RHFRed was SEQ NO: 3, and a gene sequence of the same was SEQ NO: 4.

2. Heterologous Expression and Fermentation of pET28a-CYP161H12-RHFRed

pET28a-CYP161H12-RHFRed was transformed into E. coli BL21. A single colony of BL21 harboring the heterologous expression was inoculated into 5 mL of a lysogeny broth (LB) medium and cultured at 37° C. and 220 rpm for 10 h to prepare a seed culture. The seed culture was inoculated into a sterilized terrific broth (TB) medium at an inoculation amount of 0.3%, supplemented with 50 μg/mL of kanamycin and 1 mL/L of trace metal solution (containing 50 mM of FeCl₃, 20 mM of CaCl₂, 10 mM of MnSO₄, 10 mM of ZnSO₄, 2 mM of CoSO₄, 2 mM of CuCl₂, 2 mM of NiCl₂, 2 mM of Na₂MoO₄, and 2 mM of H₃BO₃). The culture was then cultivated at 37° C. and 220 rpm until an OD value reached 0.8. The culture was cooled in an ice-water bath for 20 min, 0.1 mM of isopropyl-β-D-thiogalactopyranoside (IPTG) and 0.5 mM of 5-aminolevulinic acid hydrochloride (5-ALA) were then added to induce bacterial expression of a CYP161H12-RHFRed fusion protein. Induction was performed at 18° C. and 200 rpm for 20 h. Cells expressing CYP161H12-RHFRed were harvested by centrifugation at 3750 rpm for 5 min for subsequent in vitro catalytic reactions.

### Example 2 Expression and Fermentation of Phosphite Dehydrogenase opt13 in E. coli

1. Construction of a Recombinant Expression Vector pRSFDuet-Opt13

A pETDuet plasmid (purchased from General Biol (Anhui) Co., Ltd.) was taken as a vector. The opt13 gene (with amino acid and nucleotide sequences shown as SEQ NO: 5 and SEQ NO: 6, and synthesized by General Biol (Anhui) Co., Ltd.) was inserted into both ends of restriction endonuclease sites PstI and HindIII of the vector. Primers used were as follows: forward: TCGAGCTCGGCGCGCCTGCAGCTGCCGAAACTCGTTATAACTCAC; and reverse: GCATTATGCGGCCGCAAGCTTTTATCAGTCTGCGGCAGGATTG. The linearized vector and the PCR-cloned gene were mixed at a molar ratio of 1:2 and ligated together using a homologous recombination enzyme, and a resulting mixture was transformed into E. coli DH5a for expression. Single colonies were cultured on LB agar plate, plasmids thereof were extracted, and sequencing was performed to obtain a correctly constructed pETDuet-opt13 recombinant expression vector.

2. Heterologous Expression and Fermentation of pRSFDuet-Opt13

The pETDuet-opt13 recombinant expression vector was transformed into E. coli BL21. A single colony of BL21 harboring the heterologous expression was inoculated into 5 mL of LB medium and cultured at 37° C. and 220 rpm for 10 h to prepare a seed culture. The seed culture was inoculated into a sterilized TB medium at an inoculation amount of 0.3%, supplemented with 100 μg/mL of ampicillin. The culture was then cultivated at 37° C. and 220 rpm until an OD value reached 0.8. The culture was cooled in an ice-water bath for 20 min, 0.1 mM of isopropyl-β-D-thiogalactopyranoside (IPTG) were then added to induce bacterial expression of a protein opt13. Induction was performed at 18° C. and 200 rpm for 20 h. Cells expressing opt13 were harvested by centrifugation at 3750 rpm for 5 min for subsequent in vitro catalytic reactions.

### Example 3 Construction of a Cell-Free Catalytic System CYP161H12

The cells expressing CYP161H12-RHFRed collected in Example 1 and the cells expressing opt13 collected in Example 2 were respectively resuspended in 0.1 M KPi buffer (pH=7.0) to enable an OD₆₀₀ value to reach 30, and then sonicated to obtain cell lysates of CYP161H12-RHFRed and opt13, respectively. These cell lysates were mixed at a volume ratio of CYP161H12-RHFRed:opt13=2:1 to construct the cell-free catalytic system. CYP161H12-RHFRed served as a catalytic enzyme in the reaction.

### Example 4 Preliminary Screening and Evaluation of CYP161H12 Catalytic Activity on Different Substrates

3.33 mL of the CYP161H12-RHFRed cell lysate from Example 3 was mixed with 1.67 mL of the opt13 cell lysate, and then added to a glass tube, pre-dissolved substrates (glycyrrhetinic acid, 3-hydroxy-glycyrrhetinic acid, 3-acetyl-glycyrrhetinic acid, 3-propionyl-glycyrrhetinic acid, 3-butyryl-glycyrrhetinic acid, 3-oxo-glycyrrhetinic acid, 3-oxo-1-ene-glycyrrhetinic acid, and glycyrrhetinic acid derivatives with a seven-membered lactone ring on a ring A) in 5% (v/v) dimethyl sulfoxide (DMSO) (2 mM, 1.0 equiv.) were added together with NADP⁺ (0.5 equiv.) and Na₂HPO₃·5H₂O (50 equiv.) to the glass tube. The reaction was carried out at 23° C. and 220 rpm for 20 h to obtain a mixture. After the reaction, the mixture was extracted with 1 mL ethyl acetate and then centrifuged at 10,000 rpm for 3 min, a supernatant was collected, and an organic solvent. The remaining sample was dissolved in methanol and subjected to analysis by liquid chromatography. A protein concentration of CYP161H12-RHFRed in the reaction was 3.68×10⁻³ mM, and a catalyst dosage was 0.18 mol %.

### Example 5 Large-Scale Preparation of Enzymatic Oxidation Products

In Example 4, substrates that produced oxidized products after initial screening were scaled up according to the method in Example 3. CYP161H12-RHFRed was a fermentation cell cultured in 2 L TB medium, and opt13 was a fermentation cell cultured in 1 L TB medium. The fermentation cells were mixed to form a cell-free catalytic system for large-scale preparation. A pre-dissolved substrate in DMSO (1 equiv.), Na₂HPO₃·5H₂O (50 equiv.), and NADP⁺ (0.5 equiv.) were added, and the reaction was carried out at 23° C. and 220 rpm for 20 h to obtain a reactant. The reactant was extracted three times with ethyl acetate, concentrated, and purified to obtain the oxidation product. Specific catalytic experiments are described in the following examples.

Specifically, the TB medium was prepared as follows: 2.2 g of KH₂PO₄ and 12.3 g of K₂HPO₄·3H₂O were dissolved in 0.05 L of distilled water and autoclaved at 121° C. for 20 min as a phosphate source. 11.8 g of tryptone, 23.6 g of yeast extract, and 4 mL of glycerol were dissolved in 0.95 L of distilled water and autoclaved at 121° C. for 20 min, then cooled to 60° C., and 0.05 L of the sterilized phosphate source was added to obtain 1 L TB medium.

### Example 6 In Vitro Enzymatic Conversion for the Preparation of Oxidized Glycyrrhetinic Acid Products

Following the method described in Example 4, a yield of Substrate 1 catalyzed by CYP161H12 was determined to be as high as 100%. A ratio of hydroxylated products 1a:1b was 39:61. Following the method described in Example 5, hydroxylated products 1a and 1b were prepared using 1 g of glycyrrhetinic acid. The obtained structural data, after mass spectrometry together with proton nuclear magnetic resonance spectroscopy (¹H NMR) and Carbon-13 Nuclear Magnetic Resonance spectroscopy (¹³C NMR), are consistent with the previously reported structural data.

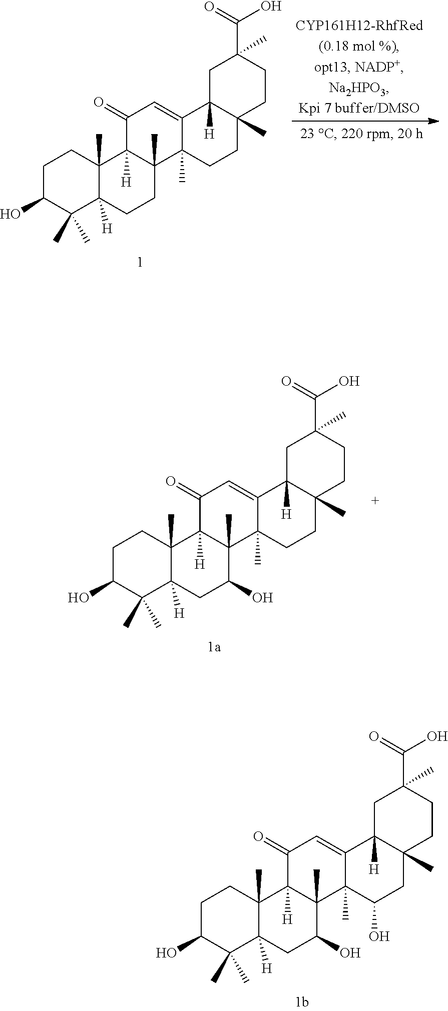

Compound 1a: ¹H NMR (400 MHz, DMSO-d6) δ_{H} 5.43 (1H, s, H-12), 3.87 (1H, dd, J=10.7, 4.7 Hz, H-3), 3.01 (1H, dd, J=4.5, 11.4 Hz, H-7), 2.26 (1H, s, H-9), 1.39, 1.10, 1.02, 1.00, 0.91, 0.76, 0.68 (s, 3H×7); ¹³C NMR (100 MHz, DMSO-d6) δ_{C} 199.1, 178.2, 170.2, 127.9, 77.0, 71.2, 61.8, 51.3, 50.4, 49.2, 44.8, 43.6, 41.3, 38.9, 37.9, 37.1, 36.7, 32.0, 30.8, 30.0, 28.9, 28.8, 28.6, 28.3, 27.5, 26.6, 23.6, 16.5, 16.4, 12.7; HRESIMS (m/z) [M+H]⁺ calculated C₃₀H₄₇O₅ 487.3423, found 487.3438.

Compound 1b: ¹H NMR (300 MHz, Pyridine-d5) δ_{H} 6.18 (1H, s, H-12), 4.68 (1H, m, H-15), 4.51 (1H, m, H-7), 3.51 (1H, m, H-3), 1.74, 1.46, 1.42, 1.35, 1.28, 1.09, 0.89 (s, 3H×7); ¹³C NMR (75 MHz, Pyridine-d5) δ_{C} 198.8, 179.0, 170.2, 129.1, 77.5, 71.4, 66.4, 62.4, 51.9, 51.7, 50.3, 49.7, 43.9, 41.3, 39.4, 39.4, 37.9, 37.7, 35.9, 32.2, 31.3, 29.2, 28.6, 28.5, 28.0, 27.8, 18.7, 16.5, 16.4, 13.2; HRESIMS (m/z) [M+H]⁺ calculated C30H₄₇O₆ 503.3373, found 503.3408.

### Example 7 Enzyme-Catalyzed Synthesis of Compounds 2a and 2b Combined with Organocatalysis

### Step A: Synthesis of Substrate 2

0.5 g (1 equiv.) of glycyrrhetinic acid was dissolved in 10 mL of acetic anhydride and heated under reflux at 130° C. for 1 h. 5 mL of acetic acid was then added. After the reaction solution was cooled, ice water was added to quench the reaction, followed by filtration to obtain a filter cake, the filter cake was then dried to obtain 0.45 g of Substrate 2 (white solid, with a yield of 82%). The obtained structural data, after mass spectrometry together with ¹H NMR and ¹³C NMR, are consistent with the previously reported structural data.

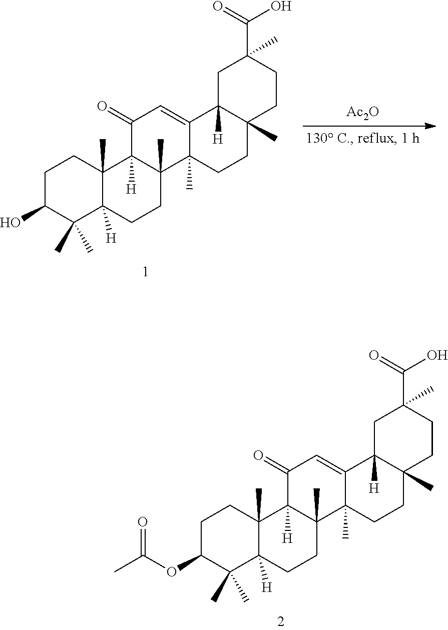

¹H NMR (400 MHz, CDCl₃): δ_{H} 5.74 (1H, s, H-12), 4.53 (1H, dd, J=4.8, 11.6 Hz, H-3), 2.39 (1H, s, H-9), 2.08 (3H, s, CH₃C═O), 1.38, 1.25, 1.18, 1.15, 0.90, 0.90, 0.86 (3H, s, CH₃×7); ¹³C NMR (101 MHz, CDCl₃): δ_{C} 200.4, 181.8, 171.1, 169.6, 128.4, 80.6, 61.7, 55.0, 48.3, 45.5, 43.8, 43.2, 40.8, 38.8, 38.1, 37.7, 36.9, 32.7, 31.9, 30.9, 28.6, 28.5, 28.1, 26.5, 26.4, 23.6, 23.4, 21.4, 18.7, 17.4, 16.7, 16.4; HRESIMS (m/z) [M+H]⁺ calculated C₃₂H₄₉O₅ 513.3580, found 513.3584.

### Step B: Enzymatic Synthesis of Compounds 2a and 2b

Following the method described in Example 4, a yield of Substrate 2 catalyzed by CYP161H12 was determined to be 92%. A ratio of Compounds 2a:2b was 91:9. Following the method described in Example 5, Compounds 2a and 2b were prepared using 0.2 g of Substrate 2. Compound 2a was analyzed by mass spectrometry, ¹H NMR and ¹³C NMR, and the obtained structural data are consistent with the previously reported structural data. Compound 2b is reported for the first time in the present disclosure.

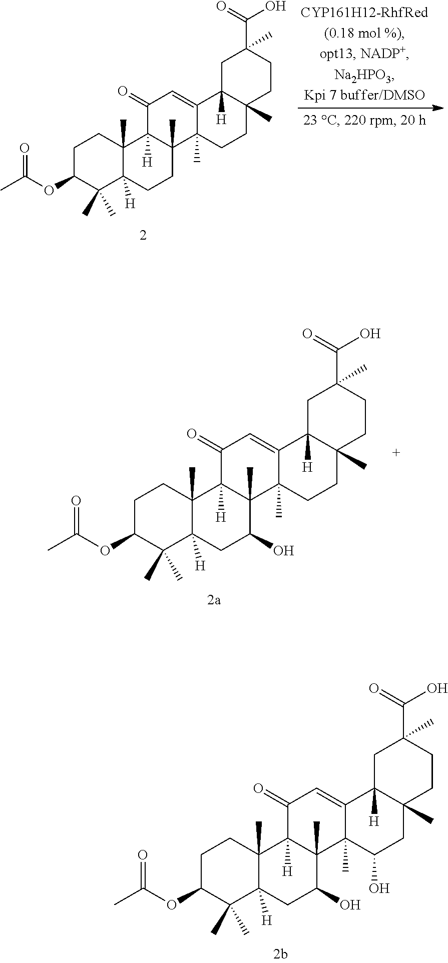

Compound 2a: ¹H NMR (400 MHz, CDCl₃): δ_{H} 5.78 (1H, s, H-12), 4.52 (1H, dd, J=4.9, 11.4 Hz, H-3), 4.10 (1H, dd, J=4.9, 10.8 Hz, H-7), 2.34 (1H, s, H-9), 2.07 (3H, s, CH₃C═O), 1.46, 1.25, 1.18, 1.16, 0.91, 0.89, 0.86 (3H, s, CH₃×7); ¹³C NMR (101 MHz, CDCl₃): δ_{C} 199.3, 181.7, 171.1, 169.3, 128.6, 80.3, 72.4, 61.9, 51.7, 50.5, 48.8, 44.5, 43.9, 41.0, 38.4, 37.8, 37.6, 37.0, 31.8, 30.9, 30.1, 28.7, 28.5, 28.5, 28.0, 26.6, 23.7, 23.5, 21.3, 16.7, 16.1, 12.3; HRESIMS (m/z) [M+H]⁺ calculated C₃₂H₄₉O₆ 529.3529, found 529.3533.

Compound 2b: ¹H NMR (400 MHz, Pyridine-d5): δ_{H} 6.13 (1H, s, H-12), 4.76 (1H, dd, J=5.2, 10.9 Hz, H-3), 4.66 (1H, m, H-15), 4.44 (1H, m, H-7), 2.55 (1H, s, H-9), 2.04 (3H, s, CH₃C═O),1.73, 1.41, 1.34, 1.33, 0.93, 0.93, 0.88 (3H, s, CH₃×7); ¹³C NMR (101 MHz, Pyridine-d5) δ_{C} 198.5, 179.1, 170.3, 170.3, 129.0, 80.1, 71.1, 66.4, 62.1, 51.7, 51.6, 50.3, 49.7, 43.8, 41.3, 38.6, 37.9, 37.8, 37.4, 35.9, 32.2, 31.3, 29.1, 28.5, 27.9, 27.3, 23.8, 20.9, 18.7, 16.8, 16.2, 13.2; HRESIMS (m/z) [M+H]⁺ calculated C₃₂H₄₉O₇ 545.3478, found 545.3478.

### Example 8 Enzyme-Catalyzed Synthesis of Compound 3a Combined with Organocatalysis

### Step A: Synthesis of Substrate 3

0.5 g (1 equiv.) of glycyrrhetinic acid was dissolved in 15 mL of anhydrous pyridine, and propionic anhydride (1.2 equiv.) and DMAP (0.2 equiv.) were then added and heated under reflux at 100° C. for 3 h. After the reaction solution was cooled, ice water was added to quench the reaction, followed by filtration to obtain a filter cake, the filter cake was then dried to obtain 0.56 g of Substrate 3 (white solid, with a yield of 99%). The obtained structural data, after mass spectrometry together with ¹H NMR and ¹³C NMR, are consistent with the previously reported structural data.

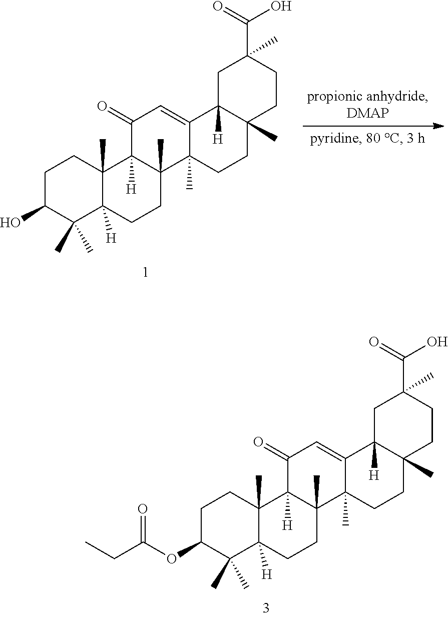

¹H NMR (300 MHz, CDCl₃): δ_{H} 5.73 (1H, s, H-12), 4.54 (1H, dd, J=5.0, 11.4 Hz, H-3), 1.39, 1.24, 1.18, 1.14, 0.89, 0.88, 0.84 (3H, s, CH₃×7); ¹³C NMR (75 MHz, CDCl₃): δ_{C} 200.4, 181.7, 174.4, 169.6, 128.4, 80.3, 61.7, 55.0, 48.2, 45.5, 43.8, 43.2, 40.9, 38.7, 38.1, 37.7, 36.9, 32.7, 31.9, 31.0, 28.5, 28.5, 28.1, 28.1, 26.5, 26.4, 23.6, 23.4, 18.7, 17.4, 16.7, 16.4, 9.4; HRESIMS (m/z) [M+H]⁺ calculated C₃₃H₅₁O₅ 527.3736, found 527.3710.

### Step B: Enzymatic Synthesis of Compound 3a

Following the method described in Example 4, a yield of Substrate 3 catalyzed by CYP161H12 was determined to be 61%. A hydroxylated product was Compound 3a. Following the method described in Example 5, Compound 3a was prepared using 0.1 g of Substrate 3. Compound 3a is reported for the first time in the present disclosure.

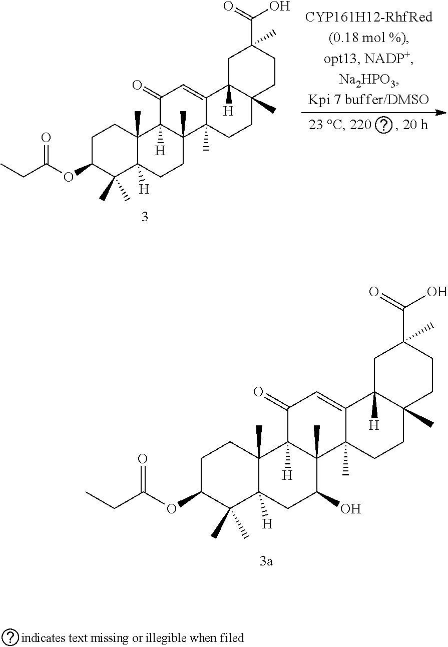

Compound 3a: ¹H NMR (300 MHz, MeOD): δ_{H} 5.65 (1H, s, H-12), 4.53 (1H, dd, J=4.7, 11.6 Hz, H-3), 4.07 (1H, dd, J=5.0, 10.8 Hz, H-7), 2.47 (1H, s, H-9), 1.19, 1.19, 1.15, 1.15, 0.94, 0.94, 0.86 (3H, s, CH₃×7); ¹³C NMR (75 MHz, MeOD) δ_{C} 200.3, 179.5, 174.6, 171.5, 127.6, 80.3, 71.5, 61.7, 51.4, 50.4, 49.2, 44.6, 43.5, 41.3, 38.0, 37.6, 37.5, 36.8, 31.5, 30.7, 29.7, 27.9, 27.8, 27.5, 27.4, 27.1, 26.3, 23.1, 22.6, 15.8, 15.3, 11.6, 8.3; HRESIMS (m/z) [M+H]⁺ calculated C₃₃H₅₁O₆ 543.3686, found 543.3724.

### Example 9 Enzyme-Catalyzed Synthesis of Compound 4a Combined with Organocatalysis

### Step A: Synthesis of Substrate 4

0.5 g (1 equiv.) of glycyrrhetinic acid was dissolved in 15 mL of anhydrous pyridine, and n-butyric anhydride (1.2 equiv.) and DMAP (0.2 equiv.) were then added and heated under reflux at 100° C. for 3 h. After the reaction solution was cooled, ice water was added to quench the reaction, followed by filtration to obtain a filter cake, the filter cake was then dried to obtain 0.33 g of Substrate 4 (white solid, with a yield of 78%). The obtained structural data, after mass spectrometry together with ¹H NMR and ¹³C NMR, are consistent with the previously reported structural data.

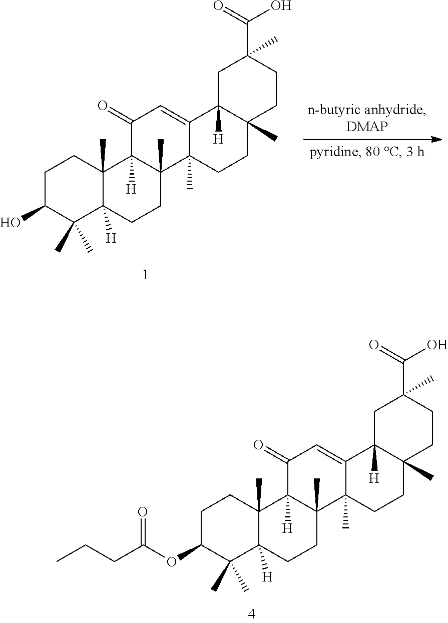

¹H NMR (400 MHz, CDCl₃): δ_{H} 5.73 (1H, s, H-12), 4.55 (1H, dd, J=4.8, 11.6 Hz, H-3), 2.39 (1H, s, H-9), 1.39, 1.24, 1.18, 1.14, 0.89, 0.89, 0.85 (3H, s, CH₃×7), 0.97 (3H, t, J=7.4 Hz, CH₃CH₂CH₂C═O); ¹³C NMR (101 MHz, CDCl₃): δ_{C} 200.5, 182.1, 173.6, 169.6, 128.4, 80.3, 61.7, 55.0, 48.2, 45.5, 43.8, 43.2, 40.8, 38.8, 38.1, 37.7, 36.9, 36.8, 32.7, 31.9, 30.9, 28.6, 28.5, 28.1, 26.5, 26.4, 23.6, 23.4, 18.7, 18.7, 17.4, 16.8, 16.4, 13.8; HRESIMS (m/z) [M+H]⁺ calculated C₃₄H₅₃O₅: 541.3893, found: 541.3894.

### Step B: Enzymatic Synthesis of Compound 4a

Following the method described in Example 4, a yield of Substrate 4 catalyzed by CYP161H12 was determined to be 23%. A hydroxylated product was Compound 4a. Following the method described in Example 5, Compound 4a was prepared using 0.1 g of Substrate 4. Compound 4a is reported for the first time in the present disclosure.

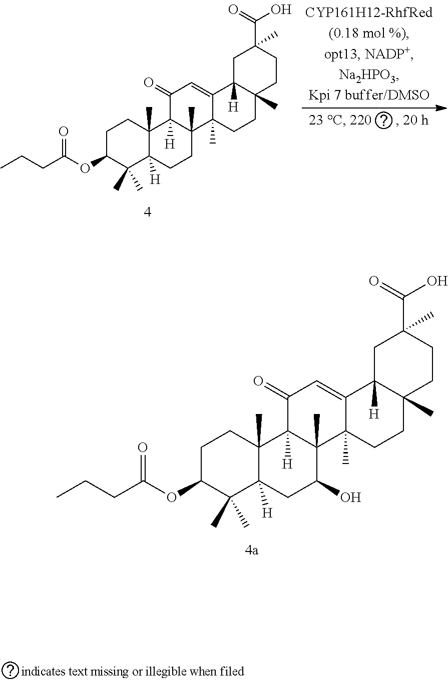

Compound 4a: ¹H NMR (400 MHz, DMSO-d6): δ_{H} 5.44 (1H, s, H-12), 4.45 (1H, dd, J=4.4, 11.8 Hz, H-3), 3.90 (1H, dd, J=4.8, 10.7 Hz, H-7), 1.40, 1.06, 1.00, 0.96, 0.83, 0.82, 0.73 (3H, s, CH₃×7), 0.89 (3H, t, J=7.3 Hz, CH₃CH₂CH₂C═O); ¹³C NMR (101 MHz, DMSO-d6): δ_{C} 199.0, 179.9, 172.9, 170.4, 127.8, 79.8, 70.8, 61.5, 50.8, 50.4, 49.3, 44.8, 43.6, 41.2, 38.02, 37.9, 37.8, 37.0, 36.3, 32.0, 30.8, 30.0, 28.8, 28.6, 28.3, 28.1, 26.6, 23.8, 23.5, 18.6, 17.1, 16.4, 13.9, 12.7; HRESIMS (m/z) [M+H]⁺ calculated C₃₄H₅₃O₆: 557.3842, found: 557.3872.

### Example 10 Enzyme-Catalyzed Synthesis of Compounds 5a and 5b Combined with Organocatalysis

### Step A: Synthesis of Substrate 5

0.5 g (1 mmol, 1 equiv.) of Compound 1 was dissolved in 5 mL of THE, placed in an ice bath, and 1.5 mL of Jones reagent was added dropwise, and then stirred for 1.5 h. A reaction solution changed from reddish-brown to clear, with dark green solid chunks formed. Ice water was added to quench the reaction, followed by filtration and drying to obtain 0.45 g of Substrate 5 (white solid, with a yield of 90%). The obtained structural data, after mass spectrometry together with ¹H NMR and ¹³C NMR, are consistent with the previously reported structural data.

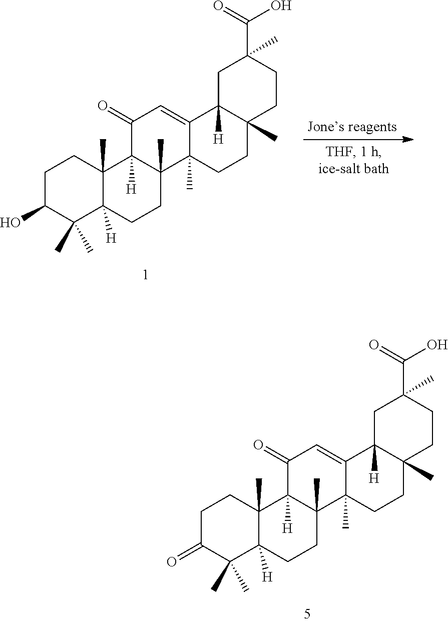

¹H NMR (400 MHz, CDCl₃): δ_{H} 5.77 (1H, s, H-12), 2.47 (1H, s, H-9), 1.40, 1.30, 1.25, 1.19, 1.13, 1.09, 0.88 (s, CH₃×7); ¹³C NMR (101 MHz, CDCl₃): δ_{C} 217.3, 199.7, 181.8, 169.9, 128.4, 61.1, 55.4, 48.3, 47.8, 45.3, 43.8, 43.3, 40.9, 39.7, 37.7, 36.7, 34.3, 32.1, 31.9, 30.9, 28.6, 28.5, 26.5, 26.4, 26.4, 23.4, 21.5, 18.8, 18.5, 15.7; LC-MS: [M−H]⁻: 467.4.

### Step B: Enzymatic Synthesis of Compounds 5a and 5b

Following the method described in Example 4, a yield of Substrate 5 catalyzed by CYP161H12 was determined to be 87%. A ratio of Compounds 5a:5b was 76:24. Following the method described in Example 5, Compounds 5a and 5b were prepared using 0.5 g of Substrate 5. Compound 5a was analyzed by mass spectrometry, ¹H NMR and ¹³C NMR, and the obtained structural data are consistent with the previously reported structural data. Compound 5b is reported for the first time in the present disclosure.

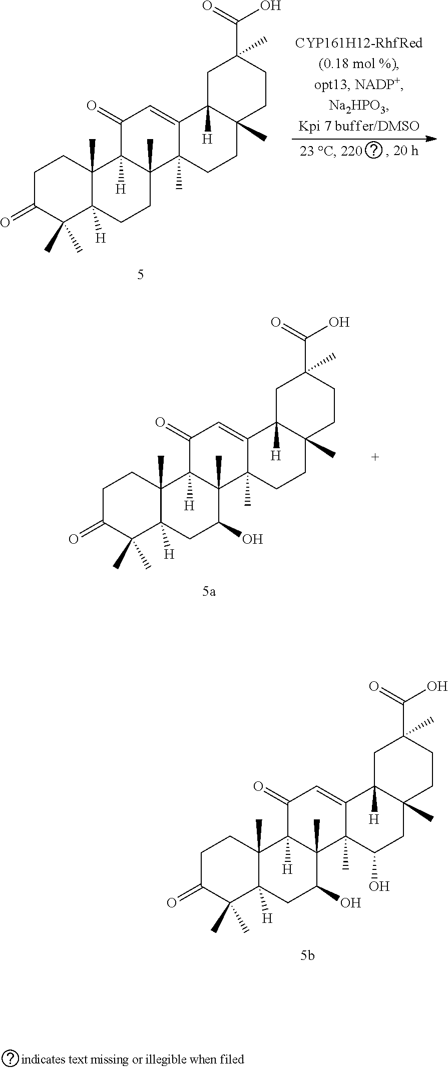

Compound 5a: ¹H NMR (400 MHz, CDCl₃): δ_{H} 5.80 (1H, s, H-12), 4.13 (1H, t, J-7.9 Hz, H-7), 2.42 (1H, s, H-9), 1.46, 1.29, 1.24, 1.20, 1.12, 1.07, 0.87 (s, CH₃×7); ¹³C NMR (101 MHz, CDCl₃): δ_{C} 216.8, 198.8, 182.0, 170.0, 128.5, 72.0, 61.1, 52.2, 50.3, 48.9, 47.4, 44.7, 43.9, 41.0, 39.3, 37.6, 36.7, 34.2, 31.8, 30.9, 30.1, 29.9, 28.6, 28.5, 26.6, 26.3, 23.59, 21.45, 15.4, 12.2; HRESIMS (m/z) [M+H]⁺ calculated C₃₀H₄₅O₅ 485.3267, found 485.3224.

Compound 5b: ¹H NMR (400 MHz, Pyridine-d5): δ_{H} 6.14 (1H, s, H-12), 4.64 (1H, dd, J=5.0, 11.4 Hz, H-15), 4.44 (1H, dd, J=5.6, 9.8 Hz, H-7), 2.64 (1H, s, H-9), 1.69, 1.41, 1.41, 1.34, 1.18, 1.07, 0.88 (s, CH₃×7); ¹³C NMR (101 MHz, Pyridine-d5) δ_{C} 215.3, 198.1, 178.9, 170.6, 128.9, 70.6, 66.5, 61.4, 51.8, 51.5, 50.3, 49.7, 47.3, 43.8, 41.3, 39.4, 37.9, 37.0, 35.9, 34.3, 32.2, 31.3, 29.1, 28.5, 26.2, 21.4, 18.7, 15.5, 13.1; HRESIMS (m/z) [M−H]⁻ calculated C₃₀H₄₃O₆ 499.3060, found 499.3060.

### Example 11 Enzyme-Catalyzed Synthesis of Compounds 6a and 6b Combined with Organocatalysis

### Step A: Synthesis of Substrate 6

2 g (4.2 mmol, 1 equiv.) of Compound 1 was dissolved in 60 mL of DMSO, and 2-iodoxybenzoic acid (4 equiv.) was added to obtain a mixture; the mixture was refluxed at 80° C. for 4 h and then cooled to room temperature; the reaction was quenched with 5% NaHCO₃ solution and extracted with diethyl ether three times. Organic layers were combined, washed with water and saturated sodium chloride solution, and dried over anhydrous sodium sulfate to remove the organic solvent, and finally eluted by 200-300 mesh silica gel column chromatography (CH₂Cl₂:MeOH=20:1) to obtain 1.77 g of Substrate 6 (white solid, with a yield of 89%). The obtained structural data, after mass spectrometry together with ¹H NMR and ¹³C NMR, are consistent with the previously reported structural data.

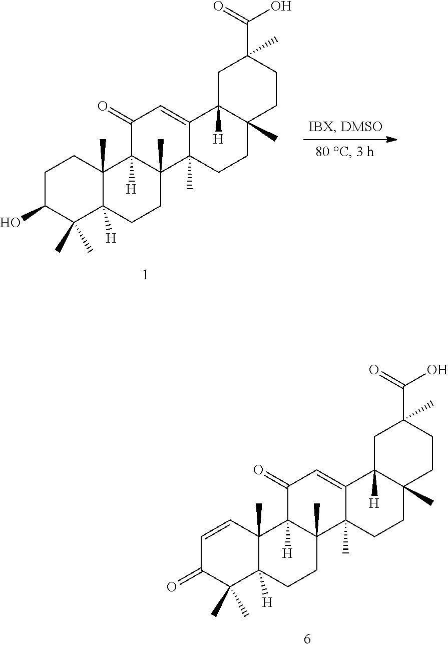

¹H NMR (400 MHz, CDCl₃): δ_{H} 7.76 (1H, d, J=10.2 Hz, H-1), 5.84 (1H, d, J=10.2 Hz, H-2), 5.83 (s, 1H, H-12), 1.44, 1.42, 1.26, 1.21, 1.18, 1.13, 0.89 (CH₃×7). ¹³C NMR (101 MHz, CDCl3): δ_{C} 204.7, 199.2, 182.2, 170.8, 161.7, 128.2, 124.6, 55.6, 52.8, 48.4, 45.6, 44.8, 43.9, 43.5, 40.9, 38.8, 37.7, 32.1, 31.9, 30.9, 28.6, 28.5, 27.6, 26.5, 26.3, 23.4, 21.6, 20.1, 19.0, 18.2. LC-MS: [M+H]⁺: 465.2.

### Step B: Enzymatic Synthesis of Compounds 6a and 6b

Following the method described in Example 4, a yield of Substrate 6 catalyzed by CYP161H12 was determined to be 81%. A ratio of hydroxylated products 6a:6b was 76:24. Following the method described in Example 5, Compounds 6a and 6b were prepared using 0.2 g of Substrate 6. Compounds 6a and 6b are reported for the first time in the present disclosure.

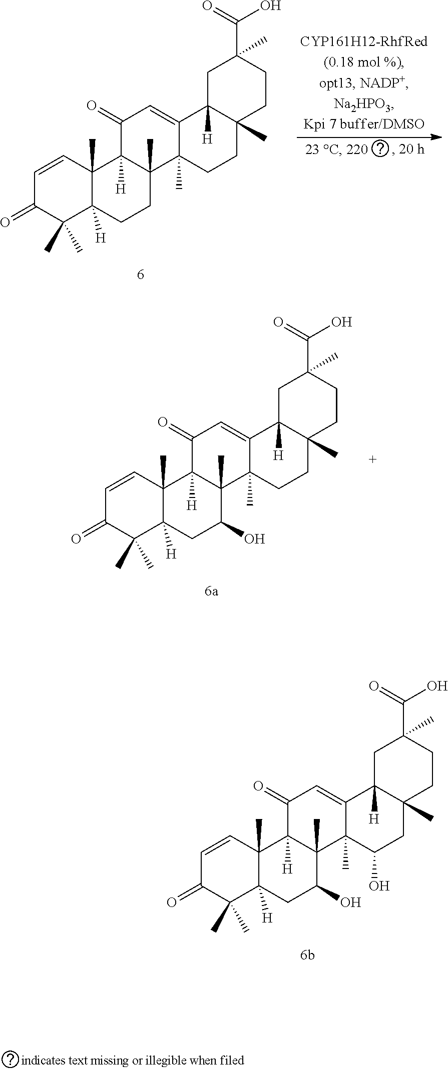

Compound 6a: ¹H NMR (400 MHz, MeOD): δ_{H} 7.75 (1H, d, J=10.2 Hz, H-1), 5.79 (1H, d, J=10.2 Hz, H-2), 5.75 (1H, s, H-12), 4.13 (1H, m, H-7), 2.83 (1H, s, H-9), 1.52, 1.43, 1.21, 1.20, 1.18, 1.13, 0.88 (s, CH₃×7); ¹³C NMR (101 MHz, MeOD) δ_{C} 205.3, 199.0, 179.1, 172.5, 162.3, 127.4, 124.0, 70.9, 55.7, 50.5, 49.4, 49.0, 44.9, 44.1, 43.6, 41.1, 38.7, 37.6, 31.5, 30.6, 29.7, 28.6, 27.9, 27.4, 26.6, 26.3, 22.5, 20.7, 19.2, 12.0; HRESIMS (m/z) [M−H]⁻ calculated C₃₀H₄₁O₅ 481.2954, found 481.2964.

Compound 6b: ¹H NMR (400 MHz, MeOD): δ_{H} 7.74 (1H, d, J=10.3 Hz, H-1), 5.80 (1H, d, J=10.4 Hz, H-2), 5.82 (1H, s, H-12), 4.34 (1H, dd, J=5.2, 11.7 Hz, H-15), 4.17 (1H, dd, J=5.2, 9.9 Hz, H-7), 2.79 (1H, s, H-9), 1.46, 1.43, 1.23, 1.21, 1.19, 1.15, 0.91 (s, CH₃×7); ¹³C NMR (75 MHz, MeOD) δ_{C} 205.2, 198.4, 179.0, 171.9, 162.2, 128.0, 124.0, 70.3, 66.3, 55.6, 51.2, 50.0, 49.6, 48.8, 44.1, 38.7, 37.3, 34.6, 31.7, 30.5, 30.4, 29.4, 28.4, 27.3, 26.8, 26.7, 20.6, 19.3, 17.2, 12.2; HRESIMS (m/z) [M−H] calculated C₃₀H₄₁O₆ 497.2903, found 497.2924.

### Example 12 Enzyme-Catalyzed Synthesis of Compounds 7a and 7b Combined with Organocatalysis

### Step A: Synthesis of Substrate 7

0.5 g (1 mmol, 1 equiv.) of Compound 5 was dissolved in 30 mL of anhydrous CH₂Cl₂, and m-chloroperoxybenzoic acid (0.175 mmol) and lithium carbonate (3.5 equiv.) were then added to obtain a mixture; the mixture was stirred at room temperature for 12 h and then quenched with 5% Na₂SO₃, and extracted with CH₂Cl₂ three times. Organic layers were combined, washed with water, saturated NaHCO₃ solution and saturated sodium chloride solution, and dried over anhydrous sodium sulfate to remove the organic solvent, and finally eluted by 200-300 mesh silica gel column chromatography (petroleum ether:ethyl acetate=2:1) to obtain 0.24 g of Substrate 7 (white solid, with a yield of 46%). The obtained structural data, after mass spectrometry together with ¹H NMR and ¹³C NMR, are consistent with the previously reported structural data.

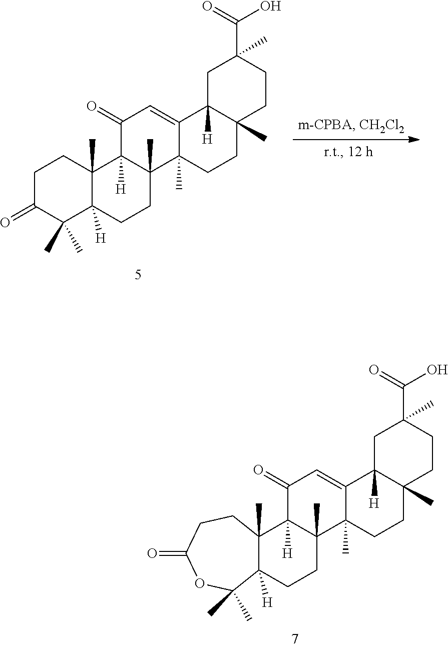

¹H NMR (400 MHz, DMSO-d6): δ_{H} 5.46 (1H, s, H-12), 1.41, 1.38, 1.34, 1.26, 1.10, 1.07, 0.77 (CH₃×7). ¹³C NMR (101 MHz, DMSO-d6): δ_{C} 198.5, 178.2, 175.0, 170.5, 127.9, 85.5, 61.2, 52.5, 49.0, 48.5, 45.3, 43.6, 43.5, 41.1, 39.5, 38.0, 32.5, 32.0, 31.8, 31.4, 30.8, 28.9, 28.3, 26.6, 26.5, 26.2, 23.3, 22.1, 18.3, 18.0. LC-MS: [M−H]⁻: 483.3.

### Step B: Enzymatic Synthesis of Compounds 7a and 7b

Following the method described in Example 4, a yield of Substrate 7 catalyzed by CYP161H12 was determined to be 87%. A ratio of hydroxylated products 7a:7b was 47:53. Following the method described in Example 5, Compounds 7a and 7b were prepared using 0.2 g of Substrate 7. Compounds 7a and 7b are reported for the first time in the present disclosure.

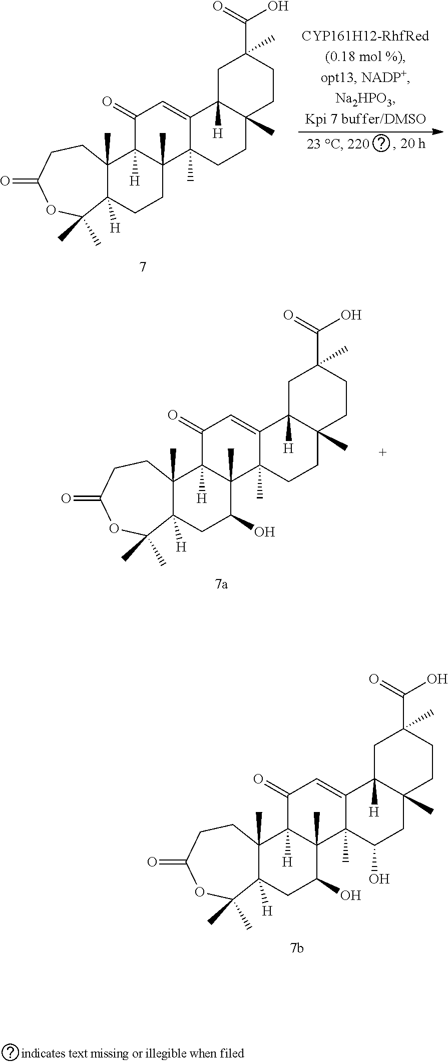

Compound 7a: ¹H NMR (300 MHz, MeOD): δ_{H} 5.70 (1H, s, H-12), 4.10 (1H, dd, J=4.4, 11.0 Hz, H-7), 5.75 (1H, s, H-12), 2.73 (1H, s, H-9), 1.53, 1.53, 1.46, 1.39, 1.19, 1.18, 0.87 (s, CH₃×7); ¹³C NMR (75 MHz, MeOD) δ_{C} 199.1, 179.2, 176.8, 171.3, 127.9, 85.5, 70.5, 61.9, 50.3, 49.9, 49.4, 44.9, 43.6, 41.2, 39.4, 39.2, 37.5, 32.5, 31.7, 31.6, 30.6, 30.2, 29.7, 27.8, 27.4, 26.3, 25.5, 22.1, 17.1, 10.8; HRESIMS (m/z) [M−H]⁻ calculated C₃₀H₄₃O₆ 499.3060, found 499.3060.

Compound 7b: ¹H NMR (300 MHz, MeOD): δ_{H} 5.77 (1H, s, H-12), 4.28 (1H, dd, J=4.4, 11.0 Hz, H-15), 4.14 (1H, dd, J=5.2, 11.6 Hz, H-7), 2.70 (1H, s, H-9), 1.54, 1.48, 1.46, 1.39, 1.20, 1.20, 0.90 (s, CH₃×7); ¹³C NMR (101 MHz, MeOD) δ_{C} 198.40, 178.9, 176.8, 170.8, 128.5, 85.4, 69.4, 66.2, 61.7, 51.2, 50.1, 49.5, 43.5, 40.8, 39.5, 39.3, 37.2, 34.7, 31.8, 31.7, 30.8, 30.5, 30.2, 29.4, 28.3, 27.3, 25.5, 17.1, 16.9, 11.0; HRESIMS (m/z) [M−H]⁻ calculated C₃₀H₄₃O₇ 515.3009, found 515.3019.

### Example 13 Enzyme-Catalyzed Synthesis of Compounds 8a and 8b Combined with Organocatalysis

### Step A: Synthesis of Substrate 8

0.5 g (1 mmol, 1 equiv.) of Compound 2 was dissolved in 8 mL of DMF, and potassium carbonate (3 equiv.) was added to obtain a mixture; the mixture was stirred at room temperature for 0.5 h, and benzyl bromide (2 equiv.) was then added dropwise, and refluxed at 100° C. for 3 h and then cooled; water was added to quench the reaction, followed by filtration to obtain a filter cake, the filter cake was then dried to obtain 0.53 g of a white solid 8-S1. Compound 8-S1 was dissolved in 8 mL of ethyl formate, and sodium hydride (16 equiv.) was added to obtain a mixture, the mixture was stirred at room temperature for 10 min, the reaction was quenched with methanol, and then extracted three times with a mixed organic solvent of CH₂Cl₂:Et₂O in a ratio of 1:2. Organic layers were combined, washed with 5% HCl, saturated NaHCO₃ solution and saturated sodium chloride solution, and dried over anhydrous sodium sulfate, and the organic solvent was removed by rotary evaporation. Elution was performed by 200-300 mesh silica gel column chromatography (petroleum ether:ethyl acetate=3:1) to obtain 0.24 g of 8-S2 (white solid, with an overall yield of the two-step reaction of 79%). 0.47 g (0.8 mmol, 1 equiv.) of Compound 8-S2 was dissolved in 10 mL of THF, 0.09 g of 10% Pd/C was then added to obtain a mixture; the mixture was stirred under a hydrogen atmosphere at room temperature for 8 h; Pd/C was removed by filtration through diatomaceous earth, and the organic solvent was removed by rotary evaporation. Elution was performed by 200-300 mesh silica gel column chromatography (CH₂Cl₂:MeOH=30:1) to obtain 0.22 g of Compound 8-S3 (white solid, with a yield of 56%). 0.22 g (0.44 mmol, 1 equiv.) of Compound 8-S3 was dissolved in 5 mL of acetic acid, and NH₂OH·HCl (5 equiv.) and anhydrous sodium acetate (0.5 equiv.) were then added to obtain a mixture; the mixture was stirred at 60° C. for 0.5 h, then cooled to room temperature; ice water was added to quench the reaction, followed by filtration to obtain a filter cake, and the filter cake was then dried, and finally eluted by 200-300 mesh silica gel column chromatography (CH₂Cl₂:MeOH=20:1) to obtain 0.17 g of Substrate 8 (white solid, with a yield of 78%). The obtained structural data, after mass spectrometry together with ¹H NMR and ¹³C NMR, are consistent with the previously reported structural data.

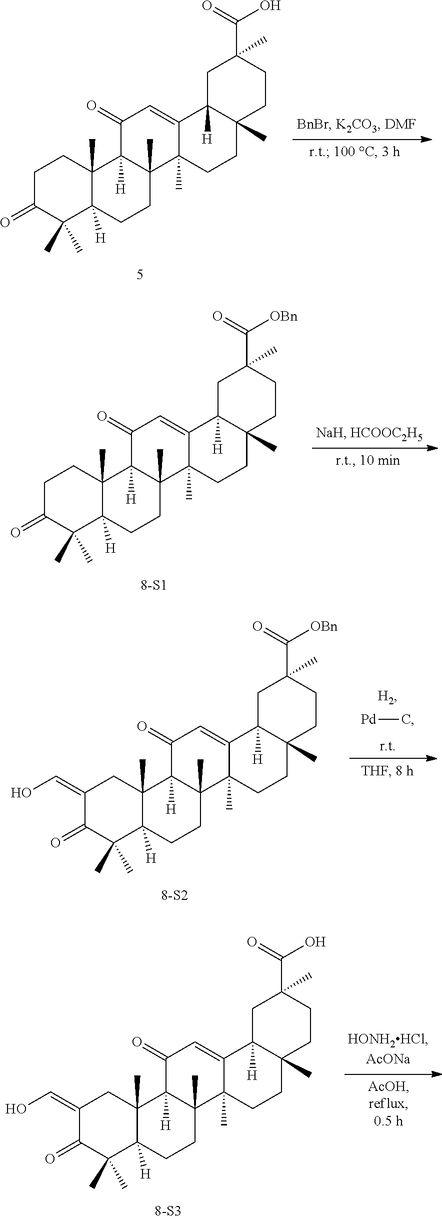

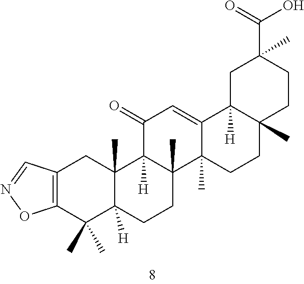

¹H NMR (300 MHz, DMSO-d6): δ_{H} 8.04 (1H, s, H-12), 2.57, 1, 1.42, 1.35, 1.27, 1.27, 1.21 (CH₃×7). ¹³C NMR (101 MHz, DMSO-d6): δ_{C} 199.7, 182.2, 172.3, 170.2, 150.4, 128.5, 109.1, 60.1, 53.3, 48.2, 45.3, 43.9, 43.4, 41.0, 38.4, 37.7, 36.0, 34.8, 31.9, 31.8, 30.9, 28.9, 28.6, 28.5, 26.6, 26.4, 23.3, 21.6, 18.3, 18.1. LC-MS: [M−H]⁻: 492.3.

### Step B: Enzymatic Synthesis of Compounds 8a and 8b

Following the method described in Example 4, a yield of Substrate 8 catalyzed by CYP161H12 was determined to be 97%. A ratio of Compounds 8a:8b was 19:81. Following the method described in Example 5, Compounds 8a and 8b were prepared using 0.2 g of Substrate 8.

Compounds 8a and 8B are reported for the first time in the present disclosure.

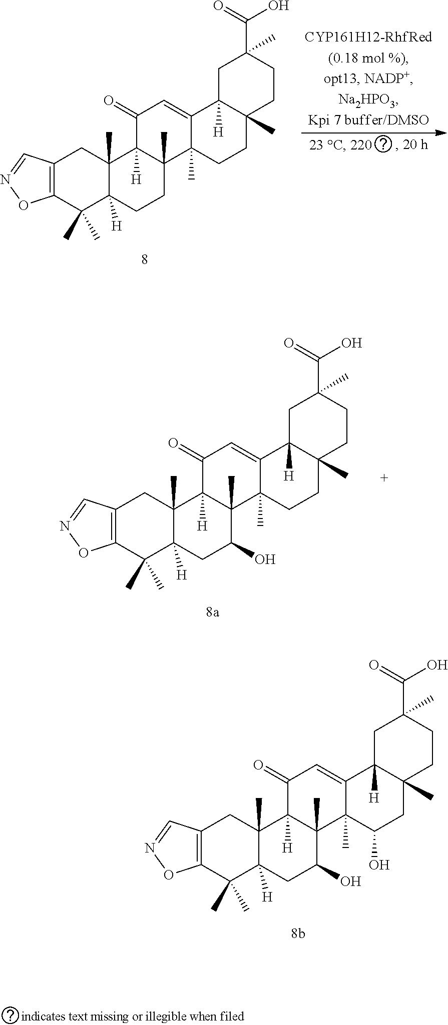

Compound 8a: ¹H NMR (300 MHz, CDCl₃): δ_{H} 8.03 (1H, s, —CH═N—), 5.85 (1H, s, H-12), 4.20 (1H, dd, J=4.7, 10.7 Hz, H-7), 2.52 (1H, s, H-9), 1.49, 1.33, 1.25, 1.25, 1.22, 1.12, 0.88 (s, CH₃×7); ¹³C NMR (75 MHz, CDCl₃) δ_{C} 198.7, 182.0, 171.9, 170.0, 150.3, 128.6, 109.2, 71.9, 60.1, 50.4, 50.3, 48.9, 44.7, 43.9, 41.1, 38.4, 37.6, 35.6, 34.6, 31.8, 30.9, 30.2, 29.2, 28.8, 28.6, 28.5, 26.6, 23.5, 21.6, 15.5, 11.9; HRESIMS (m/z) [M−H]⁻ calculated C₃₁H₄₂NO₅ 508.3063, found 508.3064.

Compound 8b: ¹H NMR (300 MHz, MeOD): δ_{H} 8.15 (1H, s, —CH═N—), 5.78 (1H, s, H-12), 4.32 (1H, dd, J=4.2, 11.7 Hz, H-15), 4.17 (1H, dd, J=4.5, 11.1 Hz, H-7), 2.64 (1H, s, H-9), 1.47, 1.36, 1.27, 1.22, 1.20, 1.12, 0.90 (s, CH₃×7); ¹³C NMR (101 MHz, MeOD) δ_{C} 198.8, 178.8, 172.0, 171.1, 150.1, 128.4, 109.4, 70.1, 66.3, 59.8, 51.1, 50.0, 49.7, 49.5, 43.5, 40.9, 38.3, 37.3, 34.9, 34.7, 34.2, 31.7, 30.6, 28.5, 27.8, 27.4, 26.8, 20.8, 17.3, 14.7, 11.6; HRESIMS (m/z) [M−H]-calculated C₃₁H₄₂NO₆ 524.3012, found 524.3023.

### Example 14 Enzyme-Catalyzed Synthesis of Compounds 9a and 9b Combined with Organocatalysis

### Step A: Synthesis of Compound 9a

The enzymatic product 1a obtained in Example 6 was used as a starting material, 0.05 g (0.1 mmol, 1 equiv.) of the enzymatic product 1a was dissolved in 2 mL of DMF, and potassium carbonate (4 equiv.) was added to obtain a mixture; the mixture was stirred at room temperature for 30 min, and iodomethane (2 equiv.) was then added. The reaction mixture was stirred at room temperature for 2.5 h, and water was then added to precipitate a large amount of white solid; the white solid was collected by filtration to obtain a filter cake, and the filter cake was then dried, and finally eluted by 200-300 mesh silica gel column chromatography (petroleum ether: ethyl acetate=1:1) to obtain 0.04 g of Compound 9a (white powder, with a yield of 78%).

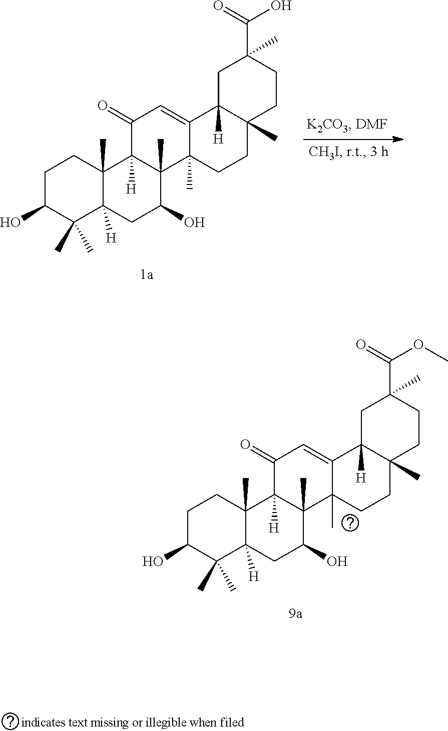

Compound 9a: ¹H NMR (300 MHz, CDCl₃): δ_{H} 5.72 (1H, s, H-12), 4.06 (1H, dd, J=5.0, 10.9 Hz, H-7), 3.24 (1H, t, J=6.8 Hz, H-3), 3.70 (3H, s, CH₃), 2.55 (1H, s, H-9), 1.45, 1.16, 1.15, 1.15, 1.03, 0.82, 0.82 (s, CH₃×7); ¹³C NMR (75 MHz, CDCl₃) δ_{C} 199.3, 177.0, 169.1, 128.6, 78.5, 72.5, 61.9, 51.8, 51.6, 50.4, 49.0, 44.5, 44.1, 41.2, 38.9, 38.8, 37.6, 37.1, 31.8, 31.1, 30.1, 28.9, 28.5, 28.4, 28.1, 27.3, 26.6, 23.7, 16.0, 15.6, 12.2; HRESIMS (m/z) [M+H]⁺ calculated C₃₁H₄₉O₅ 501.3580, found 501.3545.

### Step B: Synthesis of Compound 9b

Compound 1b obtained in Example 6 was used as a starting material, and esterification was performed according to Step A of Example 14 to obtain Compound 9b (white powder, with a yield of 49%).

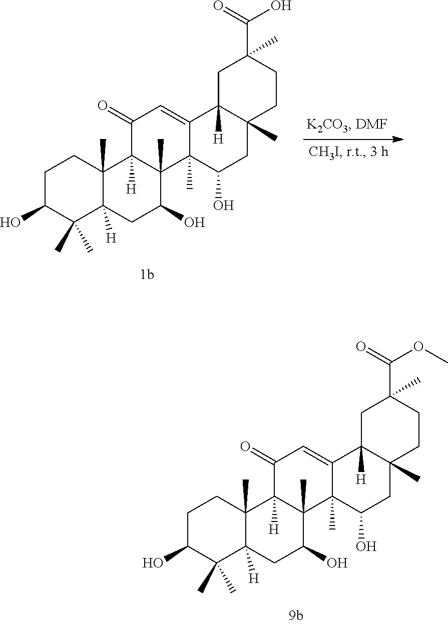

Compound 9b: ¹H NMR (400 MHz, pyridine-d5): δ_{H} 6.06 (1H, s, H-12), 4.64 (1H, dd, J=5.0, 11.5 Hz, H-15), 4.49 (1H, dd, J=4.5, 10.8 Hz, H-7), 3.66 (3H, s, OCH₃), 3.51 (1H, dd, J=4.5, 11.6 Hz, H-3), 2.48 (1H, s, H-9), 1.67, 1.44, 1.41, 1.27, 1.15, 1.08, 0.86 (s, CH₃×7); ¹³C NMR (101 MHz, pyridine-d5) δ_{C} 198.7, 176.6, 169.6, 129.1, 77.5, 71.4, 66.3, 62.4, 51.8, 51.6, 51.5, 50.3, 49.7, 44.0, 40.9, 39.3, 39.3, 37.6, 37.6, 35.8, 32.1, 31.0, 29.1, 28.5, 28.0, 27.9, 27.8, 18.6, 16.5, 16.4, 13.2; HRESIMS (m/z) [M−H]⁻ calculated C₃₁H₄₇O₆ 515.3373, found 515.3387.

### Example 15 Enzyme-Catalyzed Synthesis of Compounds 10a and 10b Combined with Organocatalysis

### Step A: Synthesis of Compound 10a

Compound 1a obtained in Example 6 was used as a starting material, 0.04 g (0.8 mmol, 1 equiv.) of Compound 1a was dissolved in 2 mL of DMF, and potassium carbonate (3 equiv.) was added to obtain a mixture; the mixture was stirred at room temperature for 30 min, and 6-bromo-1-hexene (1.5 equiv.) was then added. The reaction mixture was stirred at room temperature for 2.5 h, and water was then added to precipitate a large amount of white solid; the white solid was collected by filtration to obtain a filter cake, and the filter cake was then dried, and finally eluted by 200-300 mesh silica gel column chromatography (petroleum ether:ethyl acetate=1:1) to obtain 0.035 g of Compound 10a (white powder, with a yield of 75%).

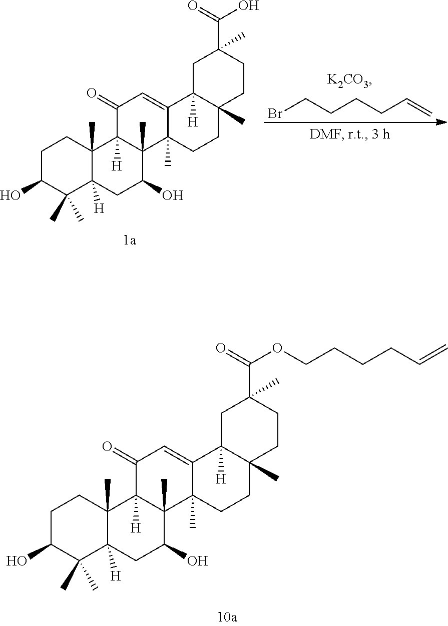

Compound 10a: ¹H NMR (400 MHz, MeOD): δ_{H} 5.04 (1H, dd, J=1.7, 17.1 Hz, CH₂═CHCH₂—), 4.98 (1H, d, J=10.2 Hz, CH₂═CHCH₂—), 4.05 (1H, dd, J=5.0, 11.0 Hz, H-7), 3.19 (1H, dd, J=4.5, 11.7 Hz, H-3), 2.42 (1H, s, H-9), 1.50, 1.17, 1.16, 1.14, 1.03, 0.85, 0.83 (s, CH₃×7); ¹³C NMR (101 MHz, MeOD): δ_{C} 200.3, 176.7, 170.9, 138.1, 127.8, 114.0, 77.8, 71.7, 64.1, 62.0, 51.5, 50.4, 49.2, 44.6, 43.9, 41.1, 38.6, 38.6, 37.5, 36.9, 33.0, 31.5, 30.6, 29.6, 28.2, 27.9, 27.8, 27.3, 27.2, 26.5, 26.3, 25.2, 22.6, 15.3, 15.0, 11.6; HRESIMS (m/z) [M+H]⁺ calculated C₃₆H₅₇O₅ 569.4206, found 569.4190.

### Step B: Synthesis of Compound 10b

Compound 1b obtained in Example 6 was used as a starting material, and esterification was performed according to Step A of Example 15 to obtain Compound 10b (white powder, with a yield of 43%).

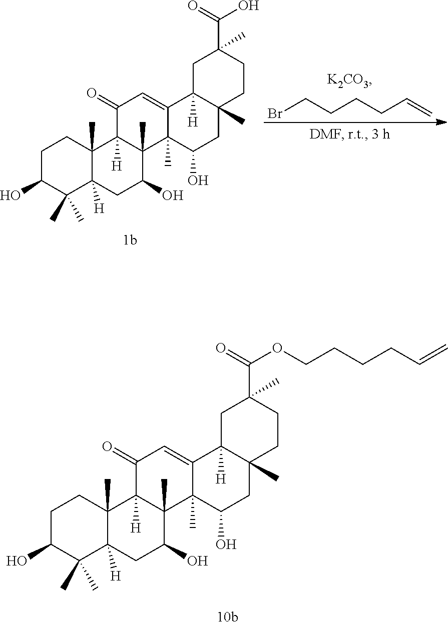

Compound 10b ¹H NMR (300 MHz, CDCl₃): δ_{H} 5.04 (1H, m, CH₂═CHCH₂—), 4.97 (1H, m, CH₂═CHCH₂—), 4.31 (1H, dd, J=4.2, 11.8 Hz, H-15), 4.07 (1H, dd, J=4.9, 11.2 Hz, H-7), 3.21 (1H, dd, J=4.6, 11.8 Hz, H-3), 2.37 (1H, s, H-9), 1.43, 1.18, 1.16, 1.16, 1.04, 0.88, 0.83 (s, CH₃×7); ¹³C NMR (101 MHz, MeOD) δ_{C} 199.5, 176.6, 170.3, 138.1, 128.4, 114.0, 77.7, 71.0, 66.2, 64.1, 61.8, 51.3, 51.2, 49.8, 49.5, 43.8, 40.7, 38.6, 38.6, 37.2, 37.1, 34.7, 33.0, 31.7, 30.5, 28.3, 27.9, 27.2, 27.2, 26.5, 26.4, 25.2, 17.2, 15.3, 15.0, 11.8; HRESIMS (m/z) [M+H]⁺ calculated C₃₆H₅₇O₆ 585.4155, found 585.4154.

### Example 16 Pharmacological Study of Products of the Present Disclosure

### In Vitro Antitumor Activity Screening Experiment

### Step A: Cell Culture

Human hepatocellular carcinoma cells (HepG2) and human colon cancer cells (SW480) were cultured in a complete culture medium consisting of high-glucose DMEM supplemented with 10% (v/v) heat-inactivated fetal bovine serum, 100 U/mL of penicillin, and 100 U/mL of streptomycin. All cells used in the experiments were cultured in an incubator at 37° C. with 5% CO₂ saturated humidity.

### Step B: Preparation of Compound Solutions at Different Concentrations

All compounds were dissolved in DMSO to prepare 20 mM compound stock solutions.
10 μL of the compound stock solution was added to 1 mL of the complete culture medium to prepare a 200 μM compound solution;
4 μL of the compound stock solution was added to 1 mL of the complete culture medium to prepare a 80 μM compound solution;
0.5 mL of the 80 μM compound solution was added to 0.5 mL of the complete culture medium to prepare a 40 μM compound solution;
0.5 mL of the 40 μM compound solution was added to 0.5 mL of the complete culture medium to prepare a 20 μM compound solution;
0.5 mL of the 20 μM compound solution was added to 0.5 mL of the complete culture medium to prepare a 10 μM compound solution;
0.5 mL of the 10 μM compound solution was added to 0.5 mL of the complete culture medium to prepare a 5 μM compound solution;

### Step C: Preliminary Screening of Cell Growth Inhibition Activity

The inhibitory effects of the compounds on tumor cell growth were evaluated using a CCK-8 assay. A cell suspension at a predetermined density (5× 104/mL cells/mL) was seeded into a 96-well plate. Outermost 36 wells of the 96-well plate were filled with 200 μL of PBS buffer (0.01 M, pH 7.4), while inner 60 wells thereof were used as a drug group, a blank group, and a negative control group. Each group was tested in triplicate.

Drug group: 100 μL of the cell suspension was added to each well and incubated for 24 h, and 100 μL of the 80 μM compound solution (100 μL of the 200 μM solution was added to Compound 1) was added for co-incubating for another 48 h; 20 μL of CCK-8 solution was added to each well, followed by incubation for another 2 h; and absorbance at 450 nm was measured with a microplate reader.

Blank group: 100 μL of the complete culture medium was added to each well and incubated for 24 h, and 100 μL of the complete culture medium was then added for co-incubating for another 48 h; 20 μL of CCK-8 solution was added to each well, followed by incubation for another 2 h; and absorbance at 450 nm was measured with a microplate reader.

Negative control group: 100 μL of the cell suspension was added to each well and incubated for 24 h, and 100 μL of the complete culture medium containing 0.4 μL of DMSO was then added for co-incubating for another 48 h; 20 μL of CCK-8 solution was added to each well, followed by incubation for another 2 h; and absorbance at 450 nm was measured with a microplate reader.

The cell growth inhibitory ratio (IR) was calculated according to the following formulas:

$$IR = 1 - ( \frac{ODpositive - ODblank}{ODnegative - ODblank} )$$

IR: Inhibitory Ratio
ODpositive: absorbance of the drug group
ODblank: absorbance of the blank group
ODnegative: absorbance of the negative control group

### Step D: Determination of the Half-Maximal Inhibitory Concentration (IC 50) of Compounds Against HepG2 and SW480 Tumor Cells

For compounds exhibiting the tumor cell inhibitory ratio greater than 50% at a concentration of 40 μM, the tumor cell inhibitory ratios at concentration gradients of 80 μM, 40 5 μM, 20 μM, 10 μM, and 5 μM were tested. Each concentration was tested in triplicate. Based on the tumor cell inhibitory ratios at different concentrations, the IC₅₀ was calculated using GraphPad Prism 9.

TABLE 1
List of IC₅₀ values of the compounds
against HepG2 and SW480 Cells (μmol/L)

IC₅₀/μM

| Compounds | HepG2 | SW480 |
|---|---|---|
| 1 | >100 | >100 |
| 1a | >40 | >40 |
| 1b | >40 | >40 |
| 2a | >40 | >40 |
| 2b | >40 | >40 |
| 3a | >40 | >40 |
| 4a | >40 | >40 |
| 5a | >40 | >40 |
| 5b | >40 | >40 |
| 6a | >40 | >40 |
| 6b | >40 | >40 |
| 7a | >40 | >40 |
| 7b | >40 | >40 |
| 8a | >40 | >40 |
| 8b | >40 | >40 |
| 9a | >40 | >40 |
| 9b | >40 | >40 |
| 10a | >40 | 39.23 ± 2.34 |
| 10b | 38.56 ± 0.54 | 32.02 ± 3.36 |

In vitro antitumor activity screening experiments of a series of glycyrrhetinic acid derivatives containing hydroxyl groups on the ring B and/or ring D provided by the present disclosure showed that Compound 10a exhibited more than twice the activity against human colon cancer cells compared with the parent compound 1 (glycyrrhetinic acid); Compound 10b showed more than twice the activity against human liver cancer cells and human colon cancer cells compared with the parent compound 1 (glycyrrhetinic acid). The above results indicate that the glycyrrhetinic acid structural skeleton obtained by selective C—H bond hydroxylation at C7β and C15α sites using the cytochrome P450 enzyme-CYP161H12 provided by the present disclosure has potential antitumor activity, and it is expected that novel antitumor drugs can be prepared by derivatization based on the hydroxylation sites.

What is claimed is:

1. A cytochrome P450 enzyme or a catalytically active fragment thereof, wherein an amino acid sequence of the same is shown in SEQ ID NO: 2.

2. A nucleic acid molecule, wherein the nucleic acid molecule encodes the cytochrome P450 enzyme or the catalytically active fragment thereof of claim 1.

3. The nucleic acid molecule according to claim 2, wherein a nucleotide sequence of the nucleic acid molecule is shown in SEQ ID NO: 1.

4. An expression cassette, a recombinant vector, and a recombinant strain or cell, comprising the nucleic acid molecule of claim 2.

5. A recombinant vector or a recombinant strain, wherein the recombinant vector is obtained by introducing the nucleic acid molecule of claim 2 into an expression vector; the expression vector comprises a plasmid pET28a-RHFRed, and the recombinant strain is obtained by introducing the recombinant vector comprising the nucleic acid molecule of claim 2 into Escherichia coli (E. coli); and the E. coli comprises E. coli BL21.

6. An enzyme-catalyzed conversion system, comprising a cell lysate 1 from the recombinant strain of claim 5; the enzyme-catalyzed conversion system further comprises a cell lysate 2 from a recombinant strain expressing phosphite dehydrogenase; and a volume ratio of the cell lysate 1 to the cell lysate 2 is 2:1.

7. A use of the cytochrome P450 enzyme or the catalytically active fragment thereof of claim 1 in the preparation of a glycyrrhetinic acid derivative.

8. A glycyrrhetinic acid derivative, wherein the glycyrrhetinic acid derivative has the following structural formulas: 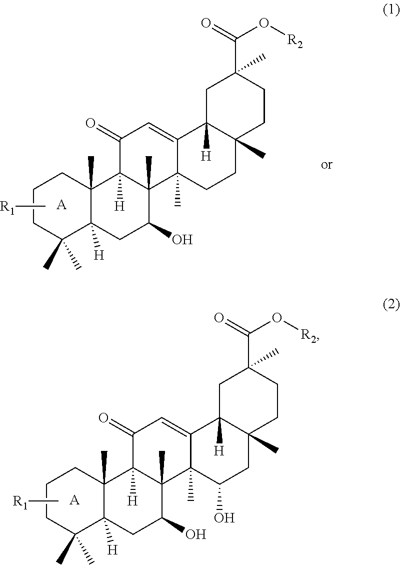
wherein R1 is a functional group on a ring A of glycyrrhetinic acid or a derivative structure based on modifications of the ring A; the functional group on the ring A comprises: a hydroxyl group at C3, an acetyl group at C3, a propionyl group at C3, a butyryl group at C3, and a ketone carbonyl group at C3; the derivative structure based on modifications of the ring A comprises an α,β-unsaturated enone of C3-oxo-C1-ene, a seven-membered lactone ring formed by ring expansion of the ring A, and fused isoxazole heterocycles at C2 and C3; and R2 comprises H, methyl or hexenyl.

9. A method for preparing the glycyrrhetinic acid derivative by combining organic synthesis and enzyme catalysis, comprising the following steps:
1) constructing a recombinant expression vector: the nucleic acid or gene of claim 2 is introduced into an expression vector to obtain the recombinant expression vector;
2) preparing and fermenting a recombinant strain: the recombinant expression vector is introduced into a host strain to obtain a recombinant strain; fermenting the recombinant strain and inducing to express, followed by ultrasonic disruption to obtain a cell lysate 1; the step further comprises fermenting and inducing expression of a recombinant strain expressing phosphite dehydrogenase, followed by ultrasonic disruption to obtain a cell lysate 2; and
3) preparing a glycyrrhetinic acid derivative by catalysing a substrate: the cell lysate 1 is reacted with the substrate to obtain the glycyrrhetinic acid derivative; it further comprises adding the cell lysate 2 for reaction; the substrate comprises glycyrrhetinic acid, 3-hydroxy-glycyrrhetinic acid, 3-acetyl-glycyrrhetinic acid, 3-propionyl-glycyrrhetinic acid, 3-butyryl-glycyrrhetinic acid, 3-oxo-glycyrrhetinic acid, 3-oxo-1-ene-glycyrrhetinic acid, and glycyrrhetinic acid derivatives with a seven-membered lactone ring on a ring A; wherein the recombinant expression vector in the step (1) comprises pET28a-CYP161H12-RHFRed; the fermentation steps in the step (2) comprises: inoculating a recombinant E. coli strain containing pET28a-CYP161H12-RHFRed into a lysogeny broth (LB) medium, and culturing at 37° C. to obtain a seed culture; adding the seed culture and a trace metal salt solution to the TB medium; culturing at 37° C. until a logarithmic growth phase occurs; then adding isopropyl-β-D-thiogalactopyranoside (IPTG) and 5-aminolevulinic acid hydrochloride (5-ALA) to induce bacterial expression of the CYP161H12-RHFRed fusion protein, followed by induction at 18° C. for 20 h; and finally collecting the cells by centrifugation for in vitro catalytic reactions; and a formulation of the trace metal salt solution is as follows: 50 mM FeCl₃, 20 mM CaCl₂), 10 mM MnSO₄, 10 mM ZnSO₄, 2 mM CoSO₄, 2 mM CuCl₂, 2 mM NiCl₂, 2 mM Na₂MoO₄, and 2 mM H₃BO₃; and the trace metal salt solution is added to the TB medium at a ratio of 0.1% v/v; a concentration of the IPTG in the TB medium is 0.1 mM, and a concentration of the 5-ALA in the TB medium is 0.5 mM; and a ratio of the cell lysate 1 to the cell lysate 2 in the step (3) is 2:1.

10. A use of the glycyrrhetinic acid derivative of claim 8 in the preparation of anti-inflammatory, antitumor, antiviral, or neuroprotective medicaments.

11. An expression cassette, a recombinant vector, and a recombinant strain or cell, comprising the nucleic acid molecule of claim 3.

12. A recombinant vector or a recombinant strain, wherein the recombinant vector is obtained by introducing the nucleic acid molecule of claim 3 into an expression vector; the expression vector comprises a plasmid pET28a-RHFRed, and the recombinant strain is obtained by introducing the recombinant vector comprising the nucleic acid molecule of claim 3 into Escherichia coli (E. coli); and the E. coli comprises E. coli BL21.

13. A use of the nucleic acid molecule of claim 2 in the preparation of a glycyrrhetinic acid derivative.

14. A use of the nucleic acid molecule of claim 3 in the preparation of a glycyrrhetinic acid derivative.

15. A use of the expression cassette, the recombinant vector, and the recombinant strain or cell of claim 4 in the preparation of a glycyrrhetinic acid derivative.

16. A use of the recombinant vector of claim 5 or the recombinant strain of claim 5 in the preparation of a glycyrrhetinic acid derivative.

17. A use of the enzyme-catalyzed conversion system of claim 6 in the preparation of a glycyrrhetinic acid derivative.

18. The glycyrrhetinic acid derivative of claim 8, wherein the glycyrrhetinic acid derivative has the following structural formulas:

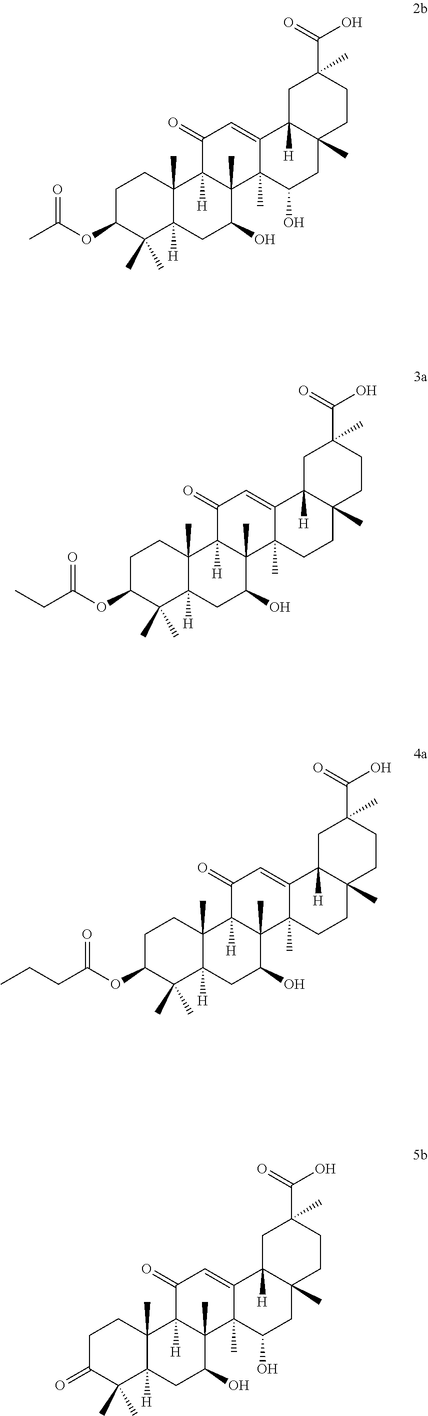

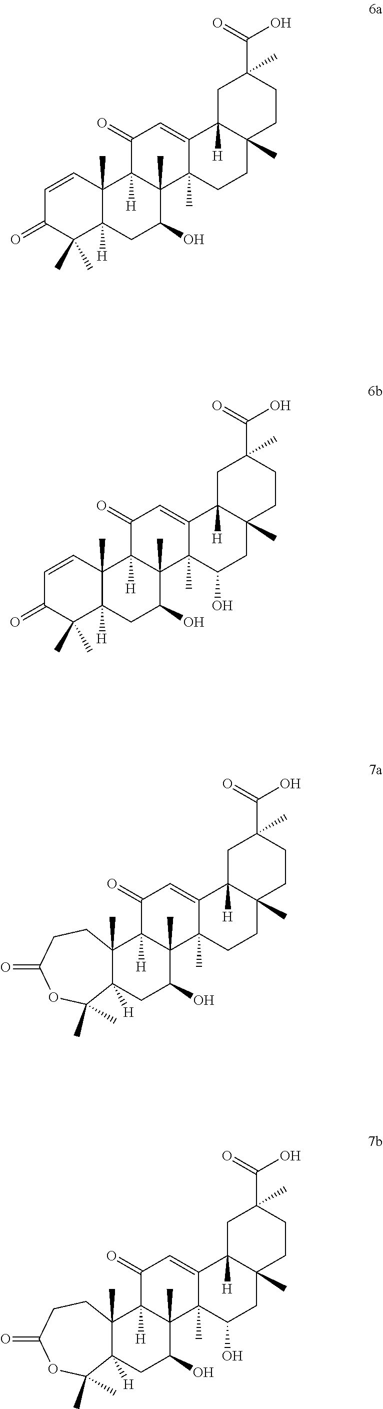

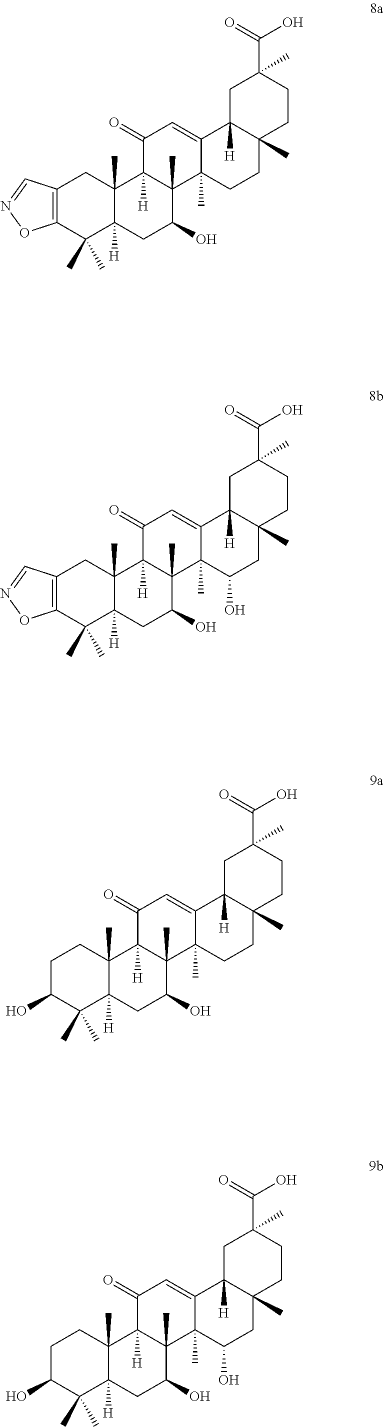

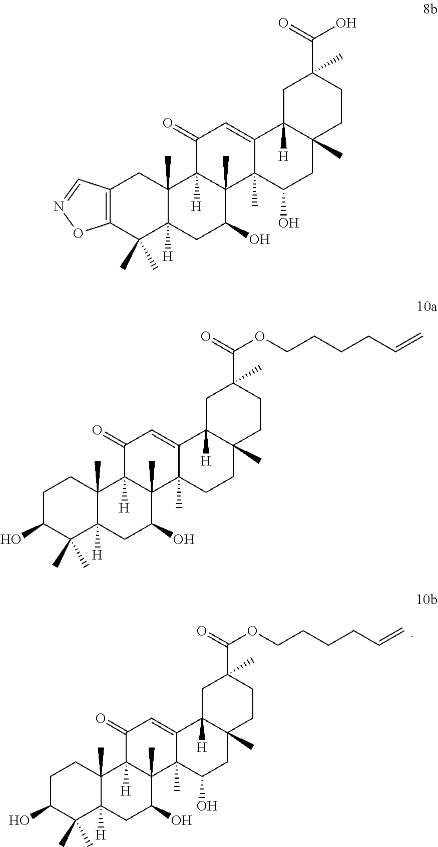

19. A method for preparing the glycyrrhetinic acid derivative by combining organic synthesis and enzyme catalysis, comprising the following steps:
1) constructing a recombinant expression vector: the nucleic acid or gene of claim 3 is introduced into an expression vector to obtain the recombinant expression vector;
2) preparing and fermenting a recombinant strain: the recombinant expression vector is introduced into a host strain to obtain a recombinant strain; fermenting the recombinant strain and inducing to express, followed by ultrasonic disruption to obtain a cell lysate 1; the step further comprises fermenting and inducing expression of a recombinant strain expressing phosphite dehydrogenase, followed by ultrasonic disruption to obtain a cell lysate 2; and
3) preparing a glycyrrhetinic acid derivative by catalysing a substrate: the cell lysate 1 is reacted with the substrate to obtain the glycyrrhetinic acid derivative; it further comprises adding the cell lysate 2 for reaction; the substrate comprises glycyrrhetinic acid, 3-hydroxy-glycyrrhetinic acid, 3-acetyl-glycyrrhetinic acid, 3-propionyl-glycyrrhetinic acid, 3-butyryl-glycyrrhetinic acid, 3-oxo-glycyrrhetinic acid, 3-oxo-1-ene-glycyrrhetinic acid, and glycyrrhetinic acid derivatives with a seven-membered lactone ring on a ring A; wherein the recombinant expression vector in the step (1) comprises pET28a-CYP161H12-RHFRed; the fermentation steps in the step (2) comprises: inoculating a recombinant E. coli strain containing pET28a-CYP161H12-RHFRed into a lysogeny broth (LB) medium, and culturing at 37° C. to obtain a seed culture; adding the seed culture and a trace metal salt solution to the TB medium; culturing at 37° C. until a logarithmic growth phase occurs; then adding isopropyl-β-D-thiogalactopyranoside (IPTG) and 5-aminolevulinic acid hydrochloride (5-ALA) to induce bacterial expression of the CYP161H12-RHFRed fusion protein, followed by induction at 18° C. for 20 h; and finally collecting the cells by centrifugation for in vitro catalytic reactions; and a formulation of the trace metal salt solution is as follows: 50 mM FeCl₃, 20 mM CaCl₂, 10 mM MnSO₄, 10 mM ZnSO₄, 2 mM CoSO₄, 2 mM CuCl₂, 2 mM NiCl₂, 2 mM Na₂MoO₄, and 2 mM H₃BO₃; and the trace metal salt solution is added to the TB medium at a ratio of 0.1% v/v; a concentration of the IPTG in the TB medium is 0.1 mM, and a concentration of the 5-ALA in the TB medium is 0.5 mM; and a ratio of the cell lysate 1 to the cell lysate 2 in the step (3) is 2:1.
